(19) United States
(12) Patent Application Publication
(10) Pub. No.: US 2011/0223187 A1
(43) Pub. Date: Sep. 15, 2011

(54) LIVE LISTERIA-BASED VACCINES FOR CENTRAL NERVOUS SYSTEM THERAPY

(76) Inventors: Vafa SHAHABI, Valley Forge, PA (US); Matthew Seavey, Prospect Park, PA (US); Yvonne Paterson, Philadelphia, PA (US)

(21) Appl. No.: 13/027,828

(22) Filed: Feb. 15, 2011

Related U.S. Application Data
(60) Provisional application No. 61/304,701, filed on Feb. 15, 2010.

(51) Int. Cl. A61K 39/02; A61P 35/00; A61P 37/04

(52) U.S. Cl. 424/192.1; 424/200.1

(57) ABSTRACT

This invention is directed to methods for treating a central nervous system (CNS) tumor or cancer using live Listeria-based recombinant vaccines.

### CROSS-REFERENCE TO RELATED APPLICATIONS

This application claims priority of U.S. Provisional Application Ser. No. 61/304,701, filed 15 Feb. 2010. This application is hereby incorporated in its entirety by reference herein.

### FIELD OF INVENTION

This invention is directed to methods for treating a central nervous system (CNS) tumor or cancer using live Listeria-based recombinant vaccines.

### BACKGROUND OF INVENTION

Listeria monocytogenes (Lm) is a gram positive facultative intracellular bacterium responsible for causing listeriosis in humans and animals. Lm is able to infect both phagocytic and non-phagocytic cells. Due to this intracellular growth behavior, Lm triggers potent innate and adaptive immune responses in an infected host that are required for the clearance of the organism. This ability, to induce efficient immune responses using multiple simultaneous and integrated mechanisms of action, has encouraged efforts to develop this bacterium as a recombinant antigen delivery vector to induce protective cellular immunity against cancer or infection.

Listeria surface proteins termed as invasins interact with receptors present on host cell plasma membranes to subvert signaling cascades leading to bacterial internalization in non-phagocytic cells. However, Listeria must escape the host cell phagolysosome to become virulent. Upon infection less than 5-8% of the Lm typically escapes into the host cell cytosol. This is mediated by Listeriolysin O (LLO), a cytolysin and the first major virulence factor of Lm identified. In the cytoplasm, Lm replicates and uses ActA, another major virulence factor, to polymerize host cell actin, support its motility and spread from cell to cell.

Hepatic Kupffer cells clear most of the circulating bacteria and are the major source of IL-6 as a consequence of LLO. Neutrophils, are rapidly recruited to the site of infection by the cytokine IL-6 and other chemo-attractants where they secrete IL-8, CSF-1, and MCP-1, which then attract macrophages to the infection foci. Granulocytes are replaced by large mononuclear cells, and within 2 weeks the lesions are completely resolved. Mice in which granulocytes are depleted are unable to survive Lm administration. Listeria replicates within hepatocytes that are then lysed by the granulocytes which migrate to the site of infection, releasing the intracellular bacteria to be phagocytosed and killed by neutrophils. Mast cells are not infected, but are activated by Lm and rapidly secrete TNF-α and induce neutrophil recruitment, and their depletion results in higher titers of Lm in liver and spleen.

Lm or sublytic doses of LLO in human epithelial Caco-2 cells induce the expression of IL-6 that reduces bacterial intracellular growth and causes over expression of inducible nitric oxide synthase (NOS). Nitric oxide appears to be an essential component of the innate immune response to Lm, having an important role in listericidal activity of neutrophils and macrophages, with a deficiency of inducible NO synthase (iNOS) causing susceptibility to Lm infection.

Lm infection also results in the generation of robust MHC Class 2 restricted CD4⁺ T cell responses, and shifts the phenotype of CD4⁺ T cells to Th-1. Further, CD4⁺ T cell help is required for the generation and maintenance of functional CD8⁺ T cell memory against Lm. Moreover, it has been reported infection of mice intraperitoneally with Lm caused a local induction of CD4⁺ T_{γδ} cells associated with IL-17 secretion in the peritoneal cavity however no changes were observed in the splenic or lymph node T cell populations after these injections. In addition, Listeria infection also involves other systems not essentially a part of the immune system but which support immune function to affect a therapeutic outcome, such as myelopoesis and vascular endothelial cell function. Innate immunity plays an essential role in the clearance of Lm and control of the infection at early stages, in fact, upon IP or IV inoculation Lm are cleared form the blood primarily by splenic and hepatic macrophages.

Lm infected macrophages produce TNF-β, IL-18 and IL-12, all of which are important in inducing the production of IFN-γ and subsequent killing and degradation of Lm in the phagosome. IL-12 deficiency results in an increased susceptibility to listeriosis, which can be reversed through administration of IFN-γ. Resistance to Lm is conferred, in part, through the release of TNF-α and IFN-γ and deficiency in either of these cytokines or their receptors increases susceptibility to Lm infection. NK cells are the major source of IFN-γ in early infection. Upon reinfection memory CD8⁺ T cells have the ability to produce IFN-γ in response to IL-12 and IL-18 in the absence of the cognate antigen. CD8⁺ T cells co-localize with the macrophages and Lm in the T cell area of the spleen where they produce IFN-γ independent of antigen. CD8⁺ T cells are also associated with Lm lesions in the liver. IFN-γ production by CD8⁺ T cells depends partially on the expression of LLO.

IFN-γ plays an important role in anti-tumor responses obtained by Lm-based vaccines. Although produced initially by NK cells, IFN-γ levels are subsequently maintained by CD4⁺ T-helper cells for a longer period. Lm vaccines require IFN-γ for effective tumor regression, and IFN-γ is specifically required for tumor infiltration of lymphocytes. IFN-γ also inhibits angiogenesis at the tumor site in the early effector phase following vaccination.

A newly observed, and as yet poorly described property of LLO, is its ability to induce epigenetic modifications affecting control of DNA expression. Extracellular LLO induces a dephosphorylation of the histone protein H3 and a similar deacetylation of the histone H4 in early phases of Listeria infection. This epigenetic effect results in reduced transcription of certain genes involved in immune function, thus providing a mechanism by which LLO may regulate the expression of gene products required for immune responses. Another genomic effect of LLO is its ability to increase NF-κβ translocation in association with the expression of ICAM and E-selectin, and the secretion of IL-8 and MCP-1. Another signaling cascade affected by LLO is the Mitogen Activated Protein Kinase (MAPK) pathway, resulting in increase of Ca²⁺ influx across the cell membrane, which facilitates the entry of Listeria into endothelial cells and their subsequent infection.

LLO is also a potent inducer of inflammatory cytokines such as IL-6, IL-8, IL-12, IL-18, TNF-α, and IFN-γ, GM-CSF as well as NO, chemokines, and costimulatory molecules that are important for innate and adaptive immune responses. The proinflammatory cytokine-inducing property of LLO is thought to be a consequence of the activation of the TLR4 signal pathway. One evidence of the high Th1 cytokine-inducing activity of LLO is in that protective immunity to Lm can be induced with killed or a virulent Lm when administered together with LLO, whereas the protection is not generated in the absence of LLO. Macrophages in the presence of LLO release IL-1α, TNF-α, IL-12 and IL-18, which in turn activate NK cells to release IFN-γ resulting in enhanced macrophage activation.

IL-18 is also critical to resistance to Lm, even in the absence of IFN-γ, and is required for TNF-α, and NO production by infected macrophages. A deficiency of caspase-1 impairs the ability of macrophages to clear Lm and causes a significant reduction in IFN-γ production and listericidal activity that can be reversed by IL-18. Recombinant IFN-γ injection restores innate resistance to listeriosis in caspase-1^{-/-} mice. Caspase-1 activation precedes the cell death of macrophages infected with Lm, and LLO deficient mutants that cannot escape the phagolysosome have an impaired ability to activate caspase-1.

LLO secreted by cytosolic Lm causes specific gene upregulation in macrophages resulting in significant IFN-γ transcription and secretion. Cytosolic LLO activates a potent type I interferon response to invasive Lm independent of Toll-like receptors (TLR) without detectable activation of NF-KB and MAPK. One of the IFN I-specific apoptotic genes, TNF-related apoptosis-inducing ligand (TRAIL), is up-regulated during Lm infection in the spleen. Mice lacking TRAIL are also more resistant to primary listeriosis coincident with lymphoid and myeloid cell death in the spleen.

The construction and development of a number of Listeria monocytogenes (Lm)-LLO based vaccines has been well documented. A variety of live, Lm strains with attenuated virulence that express viral and tumor antigens including HPV-16 E7, Her-2/neu, HMW-MAA, influenza NP, and PSA have been created to express the antigen as a fusion protein. These particular antigens can be expressed in Lm from an episomal origin, or from the Lm chromosome. Further, these recombinant strains generate profound and specific CD4⁺ and CD8⁺ T cell responses in mice. The rapid uptake of Lm into cells does not allow for humoral immunity and opsonization to develop, and this allows for repeated administration as a vaccine without loss of activity due to neutralizing humoral immune responses directed against the vector.

Live Lm-LLO vaccines are comprehensive immunotherapy and serve to effectively stimulate an immune response. For Example, Lm-LLO vaccines are cleared in SCID mice by innate immunity alone. Further, Lm-LLO vaccines effectively induce an adaptive immune response, for example, it induces high titers of CD4, CD8, APC, and tumor-infiltrating lymphocytes (TIL).

In addition to the presence of effector T cells infiltrating the tumor (TILS), other cells play an essential role in modulating the tumor microenvironment. Although current vaccines are able to induce potent T cell responses to TAAs, their therapeutic efficacy is hindered by the presence of suppressor cells in the tumor, such as regulatory T cells (Tregs), myeloid-derived suppressor cells (MDSCs) and tumor-associated macrophages (TAMs).

Live Lm-LLO vaccines also infect tumors with intra-tumoral effects as the vaccine alters tumor microenvironment that results in tumor killing, and local Mate immune effects on one hand and Tregs, MDSCs and TAMs in tumors on the other, where the latter three are a source of immune inhibition that protects tumors from immune attack. Lm-LLO vaccines modulate the tumor microenvironment, reducing the frequency of potential suppressor cells, such as MDSCs and Tregs, increasing the lymphocytic infiltration in the tumor and promoting MDSC maturation while lowering the number of MDSCs within tumors. In addition, the few cells that remain after treatment are less immunosuppressive.

Hence, there is a need for effective treatment of central nervous system (CNS) tumoral and malignant growths, and metastases thereof. Provided herein are methods and to compositions that address this need by taking advantage of the well-established therapeutic properties of live Lm-LLO vaccines to use these for the treatment of a CNS tumoral and malignant growths, and metastases thereof.

### SUMMARY OF THE INVENTION

In one embodiment, provided herein is a method of treating a growth of a central nervous system (CNS) cancer in a subject. In another embodiment, the method comprises the step of peripherally administering to a subject a composition comprising a recombinant Listeria vaccine strain comprising a nucleic acid molecule encoding a polypeptide fused to a tumor antigen. In another embodiment, the step of administering the recombinant vaccine strain results in an immune response that effects a therapeutic response across the blood brain barrier of the subject.

In another embodiment, provided herein is a method of impeding a growth of a central nervous system (CNS) cancer in a subject. In another embodiment, the method comprises the step of peripherally administering to said subject a composition comprising a recombinant Listeria vaccine strain comprising a nucleic acid molecule encoding a polypeptide fused to a tumor antigen. In another embodiment, the step of administering the recombinant vaccine strain results in an immune response that effects a therapeutic response across the blood brain barrier of the subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will be better understood from a reading of the following detailed description taken in conjunction with the drawings in which like reference designators are used to designate like elements, and in which:

FIG. 1. Expression of tLLO-WT1 fusion protein by construct LmddA174 was examined by immunobloting using anti-LLO and anti-WT1 antibody.

FIG. 2. Construction of ADXS31-164. (A) Plasmid map of pAdv164, which harbors bacillus subtilis dal gene under the control of constitutive Listeria p60 promoter for complementation of the chromosomal dal-dat deletion in LmddA strain. It also contains the fusion of truncated LLO₍₁₋₄₄₁₎ to the chimeric human Her2/neu gene, which was constructed by the direct fusion of 3 fragments the Her2/neu: EC1 (aa 40-170), EC2 (aa 359-433) and ICI (aa 678-808). (B) Expression and secretion of tLLO-ChHer2 was detected in Lm-LLO-ChHer2 and LmddA-LLO-ChHer2 (ADXS31-164) by western blot analysis of the TCA precipitated cell culture supernatants blotted with anti-LLO antibody. A differential band of ˜104 KD corresponds to tLLO-ChHer2. The endogenous LLO is detected as a 58 KD band. Listeria control lacked ChHer2 expression.

FIG. 3. Expression of tLLO-IL-13Rα2 fragments by constructs LmddA256 and LmddA257. Analysis of expression of tLLO-IL-13Rα2 fusion proteins in the culture supernatants of LmddA256 (lane 1) and LmddA257 (lane 2) by immunoblotting using LLO specific and IL-13Rα2.

FIG. 4. Western blot showing expression of tLLO-survivin fusion protein. Lane 1 represents LmddA265: tLLO-human survivin. Lane 2 represents LmddA266:tLLO-mouse survivin

FIG. 5. Shows a western blot demonstrating expression of either tLLO-CA9 or tLLO-car9 fusion proteins in the culture supernatants of LmddA181 and LmddA182 by immunoblotting using an LLO specific antibody. The bands were detected around the expected molecular size of both the fusion proteins.

FIG. 6. Shows western blot demonstrating expression of tLLO-Car9 fusion protein in the culture supernatants of LmddA181 and LmddA182 by immunoblotting with mouse car9 specific antibody. As indicated, only with LmddA182 (all three colonies) supernatants fusion protein of expected molecular size ˜89 kDa was detected.

FIG. 7. Shows western blot demonstrating expression of tLLO-CA9 fusion protein in the culture supernatants of LmddA181 and LmddA182 vaccines after immunoblotting with human CA9 specific antibody. As indicated, with only LmddA181 (all six colonies) the fusion protein of expected molecular size ˜91 kDa was detected.

FIG. 8. Shows the detection of intracellular cytokine IFN-γ after in vitro stimulation of splenocytes from control as well as LmddA174 immunized mice. The stimulation was performed using 1 μM of WT1 peptide RMFPNAPYL (SEQ ID NO: 24).

FIG. 9. Shows ELISA based detection of IFN-γ released during in vitro stimulation of LmddA174 and control Lm splenocytes in the presence of 1 μM WT1 peptide, RMFPNAPYL (SEQ ID NO: 24).

FIG. 10. Immunogenic properties of ADXS31-164. (A) Cytotoxic T cell responses elicited by Her2/neu Listeria-based vaccines in splenocytes from immunized mice were tested using NT-2 cells as stimulators and 3T3/neu cells as targets. Lm-control was based on the LmddA background that was identical in all ways but expressed an irrelevant antigen (HPV16-E7). (B) IFN-γ secreted by the splenocytes from immunized FVB/N mice into the cell culture medium, measured by ELISA, after 24 hours of in vitro stimulation with mitomycin C treated NT-2 cells. (C) IFN-γ secretion by splenocytes from HLA-A2 transgenic mice immunized with the chimeric vaccine, in response to in vitro incubation with peptides from different regions of the protein. A recombinant ChHer2 protein was used as positive control and an irrelevant peptide or no peptide groups constituted the negative controls as listed in the figure legend. IFN-γ secretion was detected by an ELISA assay using cell culture supernatants harvested after 72 hours of co-incubation. Each data point was an average of triplicate data+/−standard error. * P value<0.001.

FIG. 11. Tumor Prevention Studies for Listeria-ChHer2/neu Vaccines. Her2/neu transgenic mice were injected six times with each recombinant Listeria-ChHer2 or a control Listeria vaccine. Immunizations started at 6 weeks of age and continued every three weeks until week 21. Appearance of tumors was monitored on a weekly basis and expressed as percentage of tumor free mice. *p<0.05, N=9 per group.

FIG. 12. Effect of immunization with ADXS31-164 on the % of Tregs in Spleens. FVB/N mice were inoculated s.c. with 1×10⁶ NT-2 cells and immunized three times with each vaccine at one week intervals. Spleens were harvested 7 days after the second immunization. After isolation of the immune cells, they were stained for detection of Tregs by anti CD3, CD4, CD25 and FoxP3 antibodies. dot-plots of the Tregs from a representative experiment showing the frequency of CD25⁺/FoxP3⁺ T cells, expressed as percentages of the total CD3⁺ or CD3⁺CD4⁺ T cells across the different treatment groups.

FIG. 13. Effect of immunization with ADXS31-164 on the % of tumor infiltrating Tregs in NT-2 tumors. FVB/N mice were inoculated s.c. with 1×10⁶ NT-2 cells and immunized three times with each vaccine at one week intervals. Tumors were harvested 7 days after the second immunization. After isolation of the immune cells, they were stained for detection of Tregs by anti CD3, CD4, CD25 and FoxP3 antibodies. (A). dot-plots of the Tregs from a representative experiment. (B). Frequency of CD25⁺/FoxP3⁺ T cells, expressed as percentages of the total CD3⁺ or CD3⁺CD4⁺ T cells (left panel) and intratumoral CD8/Tregs ratio (right panel) across the different treatment groups. Data is shown as mean±SEM obtained from 2 independent experiments.

FIG. 14. Vaccination with ADXS31-164 can delay the growth of a breast cancer cell line in the brain. Balb/c mice were immunized thrice with ADXS31-164 or a control Listeria vaccine. EMT6-Luc cells (5,000) were injected intracranially in anesthetized mice. (A) Ex vivo imaging of the mice was performed on the indicated days using a Xenogen X-100 CCD camera. (B) Pixel intensity was graphed as number of photons per second per cm2 of surface area; this is shown as average radiance. (C) Expression of Her2/neu by EMT6-Luc cells, 4T1-Luc and NT-2 cell lines was detected by Western blots, using an anti-Her2/neu antibody. J774.A2 cells, a murine macrophage like cell line was used as a negative control.

### DETAILED DESCRIPTION OF THE INVENTION

In one embodiment, this invention relates to a method of treating a growth of a central nervous system (CNS) cancer using live Listeria-based vaccines. In another embodiment, live Listeria-based vaccines enable a broad number of classic and non-classic immune response mechanisms in a simultaneous and integrated manner that summates in a therapeutic effect. In another embodiment, immune responses enabled by Listeria-based vaccines include, stimulation of innate immunity sufficient to obviate the need for additional adjuvants, a strong MHC class 1 stimulation and the resultant activation of CD4⁺ cells, a strong MHC class 2 stimulation and the resultant activation of CD8⁺ cells.

In another embodiment, immune responses enabled by Listeria-based vaccines include stimulation of the bone marrow resulting in a shift toward the generation of myeloid cell lines.

In another embodiment, immune responses enabled by Listeria-based vaccines include stimulation and maturation of myeloid cells in the periphery that results in the maturation of to immature myeloid cells leading to a reduction in Myeloid Derived Suppressor Cells (MDSC).

In another embodiment, included in the number of immune responses enabled by the use of live Listeria-based vaccines is the maturation and differentiation of immature myeloid cells resulting in increases in macrophage and dendritic cell populations and their activation to a specific antigen.

In another embodiment, included in the number of immune responses enabled by the use of live Listeria-based vaccines is the stimulation of vascular endothelial cells that results in increased chemotaxis of activated immune cells and an increase in their ability to extravasate and enter tumors. Listeria monocytogenes (Lm) can invade and replicate within cultured human umbilical vein endothelial cells (HUVEC). Infection of HUVEC cells with Lm stimulates an inflammatory phenotype on these cells and induces the up-regulation of surface adhesion molecules, and in one embodiment, this event appears to be mediated by LLO. The uptake of Lm by endothelial cells provokes the signaling pathways to induce the synthesis of inflammatory chemokines and cytokines such as IL-6, IL-8, MCP-1 and GM-CSF. In another embodiment, LLO also expression of the adhesion molecules E-selectin and ICAM in human vascular endothelial cells in association with the secretion of the chemokine IL-8 and monocyte chemotaxtic protein 1 (MCP-1).

In another embodiment, the number of classic and non-classis immune responses also includes a reduction of regulatory T cells (Tregs) within tumors.

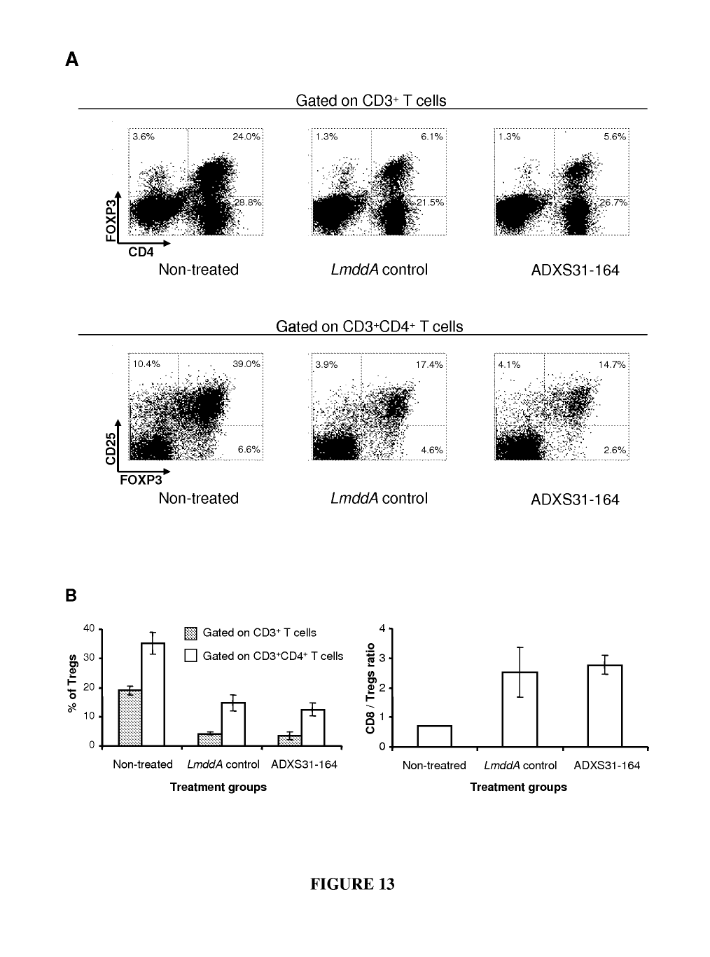

In one embodiment, the Listeria-based vaccine provided herein causes an unexpected fivefold decrease in the number of the intratumoral Tregs, as further exemplified herein (see FIG. 13).

In another embodiment, included in the number of immune responses enabled by the use of live Listeria-based vaccines are changes in the tumor microenvironment and direct effects against tumors induced by the infiltration of the live Listeria of the vaccine directly into solid tumors.

In another embodiment, included in the number of immune responses enabled by the use of live Listeria-based vaccines is the induction of epitope spreading.

In one embodiment, the above-mentioned immune response mechanisms induced by Live Listeria-based vaccines result in an unusually effective therapeutic immune response.

In one embodiment, the immune response induced by the methods and compositions provided herein is a therapeutic one. In another embodiment it is a prophylactic immune response. In another embodiment, it is an enhanced immune response over methods available in the art for inducing an immune response in a subject afflicted with the conditions provided herein. In another embodiment, the immune response leads to clearance of any disease or sequeleae that is afflicting the subject, as further provided herein.

In another embodiment, the method of treating a growth of a central nervous system (CNS) cancer in a subject comprises the step of peripherally administering to a subject a composition comprising a recombinant Listeria vaccine strain comprising a nucleic acid molecule encoding a polypeptide fused to a tumor antigen. In another embodiment, the step of administering the recombinant vaccine strain results in an immune response that effects a therapeutic response across the blood brain barrier of the subject.

In another embodiment, the invention relates to a method of impeding a growth of a CNS cancer in a subject. In another embodiment, the CNS tumor is a metastasis of a tumor originating in another part of the body. In another embodiment, the method comprises the step of peripherally administering to said subject a composition comprising a recombinant Listeria vaccine strain comprising a nucleic acid molecule encoding a polypeptide fused to a tumor antigen. In another embodiment, the step of administering the recombinant vaccine strain results in an immune response that effects a therapeutic response across the blood brain barrier of the subject.

In one embodiment, the nucleic acid molecule is in a plasmid in the recombinant Listeria vaccine strain. In another embodiment, the plasmid is stably maintained in the recombinant Listeria vaccine strain in the absence of antibiotic selection. In another embodiment, the plasmid does not confer antibiotic resistance upon said recombinant Listeria. In yet another embodiment, the nucleic acid molecule comprises a first open reading frame encoding a polypeptide, wherein said polypeptide comprises said tumor antigen. In another embodiment, the nucleic acid molecule is integrated into the Listeria genome.

In another embodiment, the nucleic acid molecule further comprises a second and a third open reading frame each encoding a metabolic enzyme. In another embodiment, the metabolic enzyme complements an endogenous gene that is lacking in the chromosome of the recombinant Listeria strain. In another embodiment, the metabolic enzyme is an amino acid metabolism enzyme. In another embodiment, the metabolic enzyme encoded by said second to open reading frame is an alanine racemase enzyme. In another embodiment, the metabolic enzyme encoded by said third open reading frame is a D-amino acid transferase enzyme.

In one embodiment, the recombinant Listeria strain is attenuated. In another embodiment, the recombinant Listeria lacks the ActA virulence gene. In another embodiment, the recombinant Listeria lacks the inlC gene. In another embodiment, the recombinant Listeria lacks the PrfA virulence gene.

In another embodiment, the live recombinant Listeria vaccine strain is a Listeria monocytogenes-LLO vaccine stain (Lm-LLO vaccine).

In one embodiment, the polypeptide encoded by the nucleic acid molecule provided herein is a fusion protein that comprises an additional polypeptide selected from the group consisting of: a) non-hemolytic LLO protein or N-terminal fragment, b) a PEST sequence, or c) an ActA fragment, and further wherein the additional polypeptide is fused to the tumor antigen. In another embodiment, the additional polypeptide is functional. In another embodiment, a fragment of the additional polypeptide is functional or is biologically active. In another embodiment, the additional polypeptide is immunogenic.

In another embodiment, the tumor antigen is PSA, HMW-MAA, HPV16-E6, HPV16-E7, VEGFR2, Her2/neu, NY-ESO1, WT-1, influenza-NP, Survivin, and IL13-R2α, CA-IX, survivin.

Anti-tumor effects have been demonstrated using murine transplantable tumors that can undergo regression after administration of Lm-based vaccines that express full antigens or fragments of antigen fused to truncated LLO.

In one embodiment, the live recombinant Listeria is used as a dual delivery vector to deliver the fusion protein comprising, for example LLO-fused to a tumor antigen that is expressed from the Listeria genome and from a plasmid to generate, for example, a simultaneous attack against a tumor antigen and an angiogenic antigen.

In another embodiment, the recombinant Listeria strain is attenuated. In another embodiment, the recombinant Listeria is attenuated for virulence. In another embodiment, the recombinant Listeria is an attenuated auxotrophic strain. In another embodiment, the high metabolic burden that the expression of a foreign antigen exerts on a bacterium such as one provided herein is also an important mechanism of attenuation. In another embodiment, the recombinant Listeria is Listeria monocytogenes.

Plasmid based strategies have the advantage of multicopy expression which are more efficacious as a function of the greater amount of antigen-LLO fusion protein expressed, but rely on complementation for the maintenance of the plasmid in vivo. Episomal expression systems are based on the fusion of a Tumor Associated Antigen (TAA) to a non-hemolytic fragment of hly (truncated LLO) that maintains the adjuvant properties of LLO.

In one embodiment, the retention of plasmid by Lm in vivo in one engineered Listeria strain is achieved by the complementation of the prfA gene from the plasmid in a prfA negative mutant Lm background. Without prfA complementation, this mutant Lm cannot escape the phagolysosome and is destroyed by macrophages and neutrophils. As a result, it cannot grow intracellularly or present antigens to the immune system. In another embodiment, including a copy of prfA in the plasmid ensures the in vivo retention of the plasmid in Lm.

In one embodiment, the retention of plasmid by Lm in vivo in an engineered Listeria strain is based on the in vitro and in vivo complementation of D-alanine racemase in both E. coli and Lm strains deficient in metabolic enzymes. In another embodiment, D-alanine racemase (dal) and D-alanine amino transferase (dat) are such enzymes. In another embodiment, the metabolic enzymes are any such enzymes available in the art. In this way, a Lm vaccine strain can be developed which is completely devoid of antibiotic selection markers.

In another embodiment, the metabolic enzyme of the methods and compositions provided herein is an amino acid metabolism enzyme, where, in another embodiment, the metabolic enzyme is an alanine racemase enzyme. In another embodiment, the metabolic enzyme is a D-amino acid transferase enzyme. In another embodiment, the metabolic enzyme catalyzes a formation of an amino acid used for a cell wall synthesis in the recombinant Listeria strain, where in another embodiment, the metabolic enzyme is an alanine racemase enzyme.

In another embodiment, the gene encoding the metabolic enzyme is expressed under the control of the Listeria p60 promoter. In another embodiment, the inlA (encodes internalin) promoter is used. In another embodiment, the hly promoter is used. In another embodiment, the ActA promoter is used. In another embodiment, the integrase gene is expressed under the control of any other gram positive promoter. In another embodiment, the gene encoding the metabolic enzyme is expressed under the control of any other promoter that functions in Listeria. The skilled artisan will appreciate that other promoters or polycistronic expression cassettes may be used to drive the expression of the gene. Each possibility represents a separate embodiment of the present invention.

“Metabolic enzyme” refers, in one embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient. Each possibility represents a separate embodiment of the present invention.

In one embodiment, fusion of LLO to tumor antigens delivered by a live Lm-based vaccine enhances their therapeutic efficacy over other vaccine modalities such as viral vectors and DNA vaccines. In another embodiment, live Lm-based vaccines have adjuvant properties that serve to enhance an immune response. In another embodiment, LLO has adjuvant properties when used in the form of a recombinant protein. In another embodiment, chemical conjugation of LLO to a tumor associated antigen (TAA) such as, but not limited to, a tumor associated antigen provided herein, induces a potent cell-mediated immune response and promotes epitope spreading after tumor challenge.

In another embodiment, the term “epitope spreading” also know as “antigen spreading” refers to the ability of the immune system to attack new targets beyond that to which the vaccine is designed to attack, by recognizing antigenic fragments of killed tumor cells as new targets. In another embodiment, Epitope spreading refers to the development of an immune response to epitopes distinct from, and noncross-reactive with, the disease-causing epitope.

Preclinical studies using a recombinant Listeria monocytogenes (Lm) strain expressing LLO-HPV-16 E7, have demonstrated both prophylactic and therapeutic efficacy against E7 expressing tumors. ADXS11-001 is an attenuated Lm which expresses and secretes a fusion protein between a truncated non-hemolytic form of Listeriolysin O and HPV16 E7. ADXS11-001 has been extensively studied in different murine HPV cancer models and was shown to eradicate 80-100% of established HPV16 E7 expressing tumors.

In one embodiment, Lm-LLO-antigen based vaccines are excellent for tumor immunotherapy because they not only induce strong T cell responses but also switch the intra-tumoral milieu from a suppressed to a less tolerant and immune active state. In one embodiment, live Listeria-based vaccines induce a potent immune response, and efficiently immunize mice against tumor proteins, overcome self-tolerance, and are safe for use in humans. The ability of Lm-based vaccines to break tolerance has been examined using transgenic mouse models for HPV-16 E6/E7 and Her-2/neu. Lm-based constructs expressing E7 fusions, such as Lm-LLO-E7 and Lm-ActA-E7, impact the growth of autochthonous tumors that arise in HPV 16 E6/E7 transgenic mice. In Her-2/neu transgenic mice which were treated with Lm-based vaccines expressing an LLO fusion with one of 5 overlapping fragments of the Her2 antigen, all the Lm-LLO-Her2 constructs were capable of slowing or halting the tumor growth or eliminating tumors, despite the fact that CD8⁺ T cells from the immunized mice were of lower avidity than those arising from wild-type mice. Lm-based Her2/neu vaccines also delayed or completely prevented the appearance of spontaneous autochthonous tumors in the transgenic Her2/neu mice. Thus, and in one embodiment, Lm based vaccines are able to overcome tolerance to self antigens and expand autoreactive T cells that are otherwise too low in number and avidity to drive anti-tumor responses. In one embodiment, the fusion of an antigen to LLO plays a major role in the induction of a CD8⁺ response as deletion of the PEST domain of LLO can cause the numbers of antigen specific CD8⁺ TILs to decrease, compromising the efficacy of the vaccine.

In one embodiment, recombinant Lm expressing TAAs fused to LLO are capable of generating potent antigen specific immune responses and provide profound anti-tumor efficacy in pre-clinical and clinical settings. In another embodiment, these immune responses can effect a therapeutic response across the blood-brain barrier.

In addition to specific anti-tumor CTL responses to the engineered antigen delivered by Listeria, immunization with attenuated Listeria can also impair the growth of tumors that do not contain epitopes present in the vaccine by, in one embodiment, a phenomenon termed epitope spreading. In one embodiment, epitope spreading (ES) is an important mechanism by which therapeutic agents can increase their efficacy by engendering immune responses to different epitopes than the target epitope(s). This effect has been associated with the use of live Listeria vaccines more than once. The phenomenon of epitope spreading occurs as a result of the release of antigens from the tumor cells killed by vaccine induced T cells, which are then phagocytosed by APCs and presented to naïve T cells in draining lymph nodes, which are then primed to respond to them. This mitigates against successful escape mutations.

In one embodiment, live Lm expressing LLO fused to a first tumor antigen induces epitope spreading to a second, endogenous tumor antigen. Anti-angiogenesis-induced tumor regression is dependent on epitope spreading to an endogenous tumor antigen. It has been demonstrated that Listeria vaccines expressing fragments of the murine VEGFR2 gene (also known as Flk-1) target tumor vasculature endothelial cells in a murine breast tumor line that over expresses Her2/neu and that immunization of mice in which these tumors are pre-established results in impaired tumor vasculature after immunization, slows or eradicates the tumors, and is accompanied by epitope spreading to the Her2/neu antigen. Hence, and in one embodiment, live Lm based vaccines, where in another embodiment, are live Lm-LLO based vaccines, induce epitope spreading and broadens the immune response to include unidentified tumor antigens in the context of therapeutic vaccines, thereby creating many more tumor targets than that for which the vaccine is engineered.

In one embodiment, recombinant attenuated, antibiotic-free Listeria expressing chimeric antigens are useful for preventing, and treating a cancer or solid tumors, as exemplified herein. In another embodiment, Lm-LLO based vaccines prevents CNS metastatic tumor formation. In another embodiment, recombinant Listeria expressing a chimeric HER2/neu is useful as a therapeutic vaccine for the treatment of Her2/neu overexpressing solid tumors in the CNS. It is to be understood that a skilled artisan can readily generate a Lm-based vaccines expressing LLO fused to a tumor antigen using methods known in the art and provided herein.

Further, and in another embodiment, a live Lm vaccine expressing LLO fused to a chimeric HER-2/neu protein induces anti-HER2/neu CTL responses in mice, shows prolonged growth stasis or eradication of Her-2/neu expressing tumors, prevents onset of tumors in Her-2/neu transgenic animals, and prevents growth of Her-2/neu expressing lung and brain tumors.

In another embodiment, PSA antigen is far more efficacious as an antigen, when delivered by Lm, as opposed to the viral vector vaccinia or as naked DNA+adjuvant. In another embodiment, Lm expressing LLO-PSA causes regression of more than 80% of tumors in a murine tumor model for PSA. Further, and in one embodiment, immunization with Lm-LLO-PSA lowers the number of tumor infiltrating T regulatory cells and causes complete regression of tumors expressing human PSA. In another embodiment, immunization with Lm-LLO-PSA leads to higher number of IFN-γ secreting cells. In another embodiment it leads to tumor regression of established tumors. In yet another embodiment, it leads to tumor regression of established tumors in a homologous prime/boost regimen.

In another embodiment, Lm targeting activated pericytes present in tumor vasculature with an LLO-HMW-MAA directed attack has potent anti-angiogenesis effects on the tumors. In another embodiment, live Lm-LLO fused to HMW-MAA fragments significantly impairs the in vivo growth of other tumorigenic cell lines, such as melanoma, renal carcinoma, and breast tumors, which were not engineered to express HMW-MAA.

In one embodiment, Lm-based vaccines induce long lasting therapeutic tumor protection against both subcutaneous tumors and metastatic tumor nodules in the lungs in melanoma models using TRP-2 as the target antigen.

In another embodiment, anti-angiogenic use of live Lm based bacteria expressing LLO induces secondary anti-tumor responses. In another embodiment, anti-angiogenic use of live Lm based bacteria expressing LLO is used for the treatment or prevention of a cancer metastasis. In another embodiment, metastatic cancer is especially susceptible to anti-angiogenesis treatment because metastases need to recruit new vessels when becoming established at distant locations from the primary tumor site. Therefore and in one embodiment, vaccination with live Lm bacteria expressing LLO can prevent the growth of metastases. In another embodiment, vaccination with live Lm bacteria expressing LLO can prevent the growth of metastases in the CNS.

In one embodiment, LLO stimulates IL-6 shifting regulatory T cells (Tregs) to Th-17 through the secretion of IL-17. In another embodiment, Tregs are another cell type that inhibit immune attacks against tumors. In another embodiment, local Lm effects include induction of CD4+ Tγδ cells associated with IL-17 secretion, where in another embodiment, it leads to a strong memory response resulting in re-induction to challenge. In one embodiment, Lm-LLO-Ag vaccines, however, decrease the population of Tregs (and possibly other inhibitory cells) and thus reduce innate immune inhibition within the tumors while simultaneously stimulating a strong attack against the tumor.

In one embodiment, Listeria and LLO stimulate lymphopoesis, monopoesis and the differentiation of undifferentiated immune cells to terminally activated effector cells.

In another embodiment, live Lm-LLO vaccines stimulate synthesis of new immune cells and maturation of existing cells. In another embodiment, live Lm-LLO vaccines stimulate chemotaxis and extravasation of activated immune cells. In another embodiment, LLO induces internalin mediated vascular endothelial invasion stimulating inflammatory chemokines and cytokines including IL-6, IL-8, MCP-1 and GM-CSF. In another embodiment, LLO transduces many events in endothelial cells in vessel endothelium such as, but not limited to, upregulation of adhesion molecules, ICAM-1, VCAM-1 and selectins and activation of NF-kB. In yet another embodiment, LLO stimulates E-selectin, ICAM, IL-8 and MCP-1 in vessel walls as well as Ca2⁺ influx.

Thus, given the potent immune response that Lm-based vaccines induce, it is to be understood that a live Listeria vaccine expressing LLO, ActA, or a PEST sequence provided herein fused to any tumor antigen such as, but not limited to those provided herein, can generate a potent immune response that can effect a therapeutic response against a CNS cancer in a subject.

The LLO utilized in the methods and compositions provided herein is, in one embodiment, a Listeria LLO. In one embodiment, the Listeria from which the LLO is derived is Listeria monocytogenes (Lm). In another embodiment, the Listeria is Listeria ivanovii. In another embodiment, the Listeria is Listeria welshimeri. In another embodiment, the Listeria is Listeria seeligeri. In another embodiment, the LLO protein is a non-Listerial LLO protein.

In one embodiment, the live Listeria recombinant vaccine strain is the ADXS31-164 strain, LmddA174 strain, LmddA256 strain, LmddA257 strain, LmddA265 strain, LmddA266 strain, the LmddA174 strain, the LmddA-181, the LmddA-182 or, as will be understood by a skilled artisan, any of the known live Listeria-based recombinant vaccines known in the art. In another embodiment, the live Listeria-based recombinant vaccine comprises an LLO protein, a PEST-sequence, and, in another embodiment, an ActA protein as further provided herein.

In one embodiment, the LLO protein is encoded by the following nucleic acid sequence set forth in (SEQ ID NO:1)

(SEQ ID NO: 1)
atgaaaaaaataatgctagtttttattacacttatattagttagtctac
caattgcgcaacaaactgaagcaaaggatgcatctgcattcaataaaga
aaattcaatttcatccatggcaccaccagcatctccgcctgcaagtcct
aagacgccaatcgaaaagaaacacgcggatgaaatcgataagtatatac
aaggattggattacaataaaaacaatgtattagtataccacggagatgc
agtgacaaatgtgccgccaagaaaaggttacaaagatggaaatgaatat
attgagtggagaaaaagaagaaatccatcaatcaaaataatgcagacat
tcaagttgtgaatgcaatttcgagcctaacctatccaggtgctctcgta
aaagcgaattcggaattagtagaaaatcaaccagatgttctccctgtaa
aacgtgattcattaacactcagcattgatttgccaggtatgactaatca
agacaataaaatagagtaaaaaatgccactaaatcaaacgttaacaacg
cagtaaatacattagtggaaagatggaatgaaaaatatgctcaagctta
tccaaatgtaagtgcaaaaattgattatgatgacgaaatggcttacagt
gaatcacaattaattgcgaaataggtacagcatttaaagctgtaaataa
tagcttgaatgtaaacttcggcgcaatcagtgaagggaaaatgcaagaa
gaagtcattagattaaacaaatttactataacgtgaatgttaatgaacc
tacaagaccaccagattatcggcaaagctgttactaaagagcagagcaa
gcgcttggagtgaatgcagaaaatcctcctgcatatatctcaagtgtgg
cgtatggccgtcaagatatttgaaattatcaactaattcccatagtact
aaagtaaaagctgatttgatgctgccgtaagcggaaaatctgtctcagg
tgatgtagaactaacaaatatcatcaaaaattcaccacaaagccgtaat
ttacggaggaccgcaaaagatgaagttcaaatcatcgacggcaacctcg
gagacttacgcgatattttgaaaaaaggcgctacttttaatcgagaaac
accaggagttcccattgcttatacaacaaacttcctaaaagacaatgaa
ttagctgttattaaaaacaactcagaatatattgaaacaacttcaaaag
cttatacagatggaaaaattaacatcgatcactctggaggatacgttgc
tcaattcaacatttcttgggatgaagtaaattatgatctcgag.

In another embodiment, the LLO protein has the sequence SEQ ID NO:2

(SEQ ID NO: 2)
M K K I M L V F I T L I L V S L P I A Q Q T E A K
D A S A F N K E N S I S S M A P P A S P P A S P K
T P I E K K H A D E I D K Y I Q G L D Y N K N N V
L V Y H G D A V T N V P P R K G Y K D G N E Y I V
V E K K K K S I N Q N N A D I Q V V N A I S S L T
Y P G A L V K A N S E L V E N Q P D V L P V K R D
S L T L S I D L P G M T N Q D N K I V V K N A T K
S N V N N A V N T L V E R W N E K Y A Q A Y P N V
S A K I D Y D D E M A Y S E S Q L I A K F G T A F
K A V N N S L N V N F G A I S E G K M Q E E V I S
F K Q I Y Y N V N V N E P T R P S R F F G K A V T
K E Q L Q A L G V N A E N P P A Y I S S V A Y G R
Q V Y L K L S T N S H S T K V K A A F D A A V S G
K S V S G D V E L T N I I K N S S F K A V I Y G G
S A K D E V Q I I D G N L G D L R D I L K K G A T
F N R E T P G V P I A Y T T N F L K D N E L A V I
K N N S E Y I E T T S K A Y T D G K I N I D H S G
G Y V A Q F N I S W D E V N Y D L

The first 25 amino acids of the proprotein corresponding to this sequence are the signal sequence and are cleaved from LLO when it is secreted by the bacterium. Thus, in this embodiment, the full length active LLO protein is 504 residues long. In another embodiment, the LLO protein has a sequence set forth in GenBank Accession No. DQ054588, DQ054589, AY878649, U25452, or U25452. In another embodiment, the LLO protein is a variant of an LLO protein. In another embodiment, the LLO protein is a homologue of an LLO protein. Each possibility represents a separate embodiment of the present invention.

In another embodiment, “truncated LLO” or “tLLO” refers to a fragment of LLO that comprises the PEST-like domain. In another embodiment, the terms refer to an LLO fragment that does not contain the activation domain at the amino terminus and does not include cystine 484. In another embodiment, the LLO fragment consists of a PEST sequence. In another embodiment, the LLO fragment comprises a PEST sequence. In another embodiment, the LLO fragment consists of about the first 400 to 441 amino acids of the 529 amino acid full-length LLO protein. In another embodiment, the LLO fragment is a non-hemolytic form of the LLO protein.

In one embodiment, the LLO fragment consists of about residues 1-25. In another embodiment, the LLO fragment consists of about residues 1-50. In another embodiment, the LLO fragment consists of about residues 1-75. In another embodiment, the LLO fragment consists of about residues 1-100. In another embodiment, the LLO fragment consists of about residues 1-125. In another embodiment, the LLO fragment consists of about residues 1-140. In another embodiment, the LLO fragment consists of about residues 1175. In another embodiment, the LLO fragment consists of about residues 1-200. In another embodiment, the LLO fragment consists of about residues 1-225. In another embodiment, the LLO fragment consists of about residues 1-250. In another embodiment, the LLO fragment consists of about residues 1-275. In another embodiment, the LLO fragment consists of about residues 1-300. In another embodiment, the LLO fragment consists of about residues 1-325. In another embodiment, the LLO fragment consists of about residues 1-350. In another embodiment, the LLO fragment consists of about residues 1-375. In another embodiment, the LLO fragment consists of about residues 1-400. In another embodiment, the LLO fragment consists of about residues 1-425. Each possibility represents a separate embodiment of the present invention.

In another embodiment, a fusion protein of methods and compositions of the present invention comprises a PEST sequence, either from an LLO protein or from another organism, e.g. a prokaryotic organism.

The PEST-like AA sequence has, in another embodiment, a sequence selected from SEQ ID NO: 3-7. In another embodiment, the PEST-like sequence is a PEST-like sequence from the Lm ActA protein. In another embodiment, the PEST-like sequence is KTEEQPSEVNTGPR (SEQ ID NO: 3), KASVTDTSEGDLDSSMQSADESTPQPLK (SEQ ID NO: 4), KNEEVNASDFPPPPTDEELR (SEQ ID NO: 5), or RGGIPTSEEFSSLNSGDFTDDENSETTEEEIDR (SEQ ID NO: 6). In another embodiment, the PEST-like sequence is from Streptolysin O protein of Streptococcus sp. In another embodiment, the PEST-like sequence is from Streptococcus pyogenes Streptolysin O, e.g. KQNTASTETTTTNEQPK (SEQ ID NO: 7) at AA 35-51. In another embodiment, the PEST-like sequence is from Streptococcus equisimilis Streptolysin O, e.g. KQNTANTETTTTNEQPK (SEQ ID NO: 8) at AA 38-54. In another embodiment, the PEST-like sequence is another PEST-like AA sequence derived from a prokaryotic organism. In another embodiment, the PEST-like sequence is any other PEST-like sequence known in the art. Each possibility represents a separate embodiment of the present invention.

In one embodiment, fusion of an antigen to the PEST-like sequence of Lm enhanced cell mediated and anti-tumor immunity of the antigen. Thus, fusion of an antigen to other PEST-like sequences derived from other prokaryotic organisms will also enhance immunogenicity of the antigen. PEST-like sequence of other prokaryotic organism can be identified in accordance with methods such as described by, for example Rechsteiner and Rogers (1996, Trends Biochem. Sci. 21:267-271) for Lm. Alternatively, PEST-like AA sequences from other prokaryotic organisms can also be identified based by this method. Other prokaryotic organisms wherein PEST-like AA sequences would be expected to include, but are not limited to, other Listeria species. In another embodiment, the PEST-like sequence is embedded within the antigenic protein. Thus, in another embodiment, “fusion” refers to an antigenic protein comprising both the antigen and the PEST-like amino acid sequence either linked at one end of the antigen or embedded within the antigen.

In another embodiment, “LLO fragment” or “ΔLLO” refers to a fragment of LLO that comprises the PEST-like domain thereof. In another embodiment, the terms refer to an LLO fragment that comprises a PEST sequence. Each possibility represents another embodiment of the present invention.

In another embodiment, the LLO fragment is any other LLO fragment known in the art. Each possibility represents a separate embodiment of the present invention.

In another embodiment, a whole LLO protein is utilized in methods and compositions of the present invention. In another embodiment, the whole LLO protein is a non-hemolytic LLO protein.

The tumor antigen of methods and compositions of the present invention is, in another embodiment, a heterologous antigenic polypeptide or functional fragment thereof, which is in another embodiment, an antigenic protein. In another embodiment, an antigenic peptide is a fragment of an antigenic protein. In another embodiment, the antigenic peptide is an immunogenic peptide derived from tumor. In another embodiment, the antigenic peptide is an immunogenic peptide derived from metastasis. In another embodiment, the antigenic peptide is an immunogenic peptide derived from cancerous cells. In another embodiment, the antigenic peptide is a pro-angiogenesis immunogenic peptide. In another embodiment, the heterologous antigenic peptide is a tumor-associated antigen (TAA).

In another embodiment, the antigenic polypeptide is Human Papilloma Virus-E7 (HPV-E7) antigen, which in one embodiment, is from HPV16 (in one embodiment, GenBank Accession No. AAD33253) and in another embodiment, from HPV18 (in one embodiment, GenBank Accession No. P06788). In another embodiment, the antigenic polypeptide is HPV-E6, which in one embodiment, is from HPV16 (in one embodiment, GenBank Accession No. AAD33252, AAM51854, AAM51853, or AAB67614) and in another embodiment, from HPV18 (in one embodiment, GenBank Accession No. P06463). In another embodiment, the antigenic polypeptide is a Her/2-neu antigen. In another embodiment, the antigenic polypeptide is Prostate Specific Antigen (PSA) (in one embodiment, GenBank Accession No. CAD30844, CAD54617, AAA58802, or NP_{—}001639). In another embodiment, the antigenic polypeptide is Stratum Corneum Chymotryptic Enzyme (SCCE) antigen (in one embodiment, GenBank Accession No. AAK69652, AAK69624, AAG33360, AAF01139, or AAC37551). In another embodiment, the antigenic polypeptide is Wilms tumor antigen 1, which in another embodiment is WT-1 Telomerase (GenBank Accession. No. P49952, P22561, NP_{—}659032, CAC39220.2, or EAW68222.1). In another embodiment, the antigenic polypeptide is hTERT or Telomerase (GenBank Accession. No. NM003219 (variant 1), NM198255 (variant 2), NM 198253 (variant 3), or NM 198254 (variant 4). In another embodiment, the antigenic polypeptide is Proteinase 3 (in one embodiment, GenBank Accession No. M29142, M75144, M96839, X55668, NM 00277, M96628 or X56606). In another embodiment, the antigenic polypeptide is Tyrosinase Related Protein 2 (TRP2) (in one embodiment, GenBank Accession No. NP_{—}001913, ABI73976, AAP33051, or Q95119). In another embodiment, the antigenic polypeptide is High Molecular Weight Melanoma Associated Antigen (HMW-MAA) (in one embodiment, GenBank Accession No. NP_{—}001888, AAI28111, or AAQ62842). In another embodiment, the antigenic polypeptide is Testisin (in one embodiment, GenBank Accession No. AAF79020, AAF79019, AAG02255, AAK29360, AAD41488, or NP_{—}659206). In another embodiment, the antigenic polypeptide is NY-ESO-1 antigen (in one embodiment, GenBank Accession No. CAA05908, P78358, AAB49693, or NP_{—}640343). In another embodiment, the antigenic polypeptide is PSCA (in one embodiment, GenBank Accession No. AAH65183, NP_{—}005663, NP_{—}082492, O43653, or CAB97347). In another embodiment, the antigenic polypeptide is Interleukin (IL) 13 Receptor alpha (in one embodiment, GenBank Accession No. NP_{—}000631, NP_{—}001451, NP_{—}032382, NP_{—}598751, NP_{—}001003075, or NP_{—}999506). In another embodiment, the antigenic polypeptide is Carbonic anhydrase IX (CAIX) (in one embodiment, GenBank Accession No. CAI13455, CAI10985, EAW58359, NP_{—}001207, NP_{—}647466, or NP_{—}001101426). In another embodiment, the antigenic polypeptide is carcinoembryonic antigen (CEA) (in one embodiment, GenBank Accession No. AAA66186, CAA79884, CAA66955, AAA51966, AAD14250, or AAA51970). In another embodiment, the antigenic polypeptide is MAGE-A (in one embodiment, GenBank Accession No. NP_{—}786885, NP_{—}786884, NP_{—}005352, NP_{—}004979, NP_{—}005358, or NP_{—}005353). In another embodiment, the antigenic polypeptide is survivin (in one embodiment, GenBank Accession No. AAC51660, AAY14202, ABF60110, NP_{—}001003019, or NP_{—}001082350). In another embodiment, the antigenic polypeptide is GP100 (in one embodiment, GenBank Accession No. AAC60634, YP_{—}655861, or AAB31176). In another embodiment the antigen is carbo-anhydrase 9 (CA-9). In another embodiment, the antigenic polypeptide is any other antigenic polypeptide known in the art. In another embodiment, the antigenic peptide of the compositions and methods of the present invention comprise an immunogenic portion of the antigenic polypeptide. Each possibility represents a separate embodiment of the present invention.

In another embodiment, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, HIV env protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, human papilloma virus antigens E1 and E2 from type HPV-16, -18, -31, -33, -35 or -45 human papilloma viruses, the tumor antigens CEA, the ras protein, mutated or otherwise, the p53 protein, mutated or otherwise.

In various embodiments, the antigen of methods and compositions of the present invention includes but is not limited to antigens or functional fragments thereof from the following infectious diseases, measles, mumps, rubella, poliomyelitis, hepatitis A, B (e.g., GenBank Accession No. E02707), and C (e.g., GenBank Accession No. E06890), as well as other hepatitis viruses, type A influenza, other types of influenza, adenovirus (e.g., types 4 and 7), rabies (e.g., GenBank Accession No. M34678), yellow fever, Japanese encephalitis (e.g., GenBank Accession No. E07883), dengue (e.g., GenBank Accession No. M24444), hantavirus, and HIV (e.g., GenBank Accession No. U18552). Bacterial and parasitic antigens will be derived from known causative agents responsible for diseases including, but not limited to, diphtheria, pertussis (e.g., GenBank Accession No. M35274), tetanus (e.g., GenBank Accession No. M64353), tuberculosis, bacterial and fungal pneumonias (e.g., Haemophilus influenzae, Pneumocystis carinii, etc.), cholera, typhoid, plague, shigellosis, salmonellosis (e.g., GenBank Accession No. L03833), Legionnaire's Disease, Lyme disease (e.g., GenBank Accession No. U59487), malaria (e.g., GenBank Accession No. X53832), hookworm, onchocerciasis (e.g., GenBank Accession No. M27807), schistosomiasis (e.g., GenBank Accession No. L08198), trypanosomiasis, leshmaniasis, giardiasis (e.g., GenBank Accession No. M33641), amoebiasis, filariasis (e.g., GenBank Accession No. J03266), borreliosis, and trichinosis.

In other embodiments, the antigen is one of the following tumor antigens or a functional fragment thereof: any of the various MAGEs (Melanoma-Associated Antigen E), including MAGE 1 (e.g., GenBank Accession No. M77481), MAGE 2 (e.g., GenBank Accession No. U03735), MAGE 3, MAGE 4, TRP-2, gp-100, tyrosinase, MART-1, HSP-70, and beta-HCG; a tyrosinase; mutant ras; mutant p53 (e.g., GenBank Accession No. X54146 and AA494311); and p97 melanoma antigen (e.g., GenBank Accession No. M12144). Other tumor-specific antigens include the Ras peptide and p53 peptide associated with advanced cancers, the HPV 16/18 and E6/E7 antigens associated with cervical cancers, MUC1 antigen associated with breast carcinoma (e.g., GenBank Accession No. J0365 1), CEA (carcinoembryonic antigen) associated with colorectal cancer (e.g., GenBank Accession No. X983 11), gp100 (e.g., GenBank Accession No. 573003) or MARTI antigens associated with melanoma, and the prostate-specific antigen (KLK3) associated with prostate cancer (e.g., GenBank Accession No. X14810). The p53 gene sequence is known (See e.g., Harris et al. (1986) Mol. Cell. Biol., 6:4650-4656) and is deposited with GenBank under Accession No. M14694. Tumor antigens encompassed by the present invention further include, but are not limited to, Her-2/Neu (e.g. GenBank Accession Nos. M16789.1, M16790.1, M16791.1, M16792.1), NY-ESO-1 (e.g. GenBank Accession No. U87459), WT-1 (e.g. GenBank Accession Nos. NM000378 (variant A), NM024424 (variant B), NM 024425 (variant C), and NM024426 (variant D)), LAGE-1 (e.g. GenBank Accession No. CAA11044), synovial sarcoma, X (SSX)-2; (e.g. GenBank Accession No. NP_{—}003138, NP_{—}783629, NP_{—}783729, NP_{—}066295), and stratum corneum chymotryptic enzyme (SCCE; GenBank Accession No. NM_{—}005046 and NM_{—}139277)). Thus, the present invention can be used as immunotherapeutics for cancers including, but not limited to, cervical, breast, colorectal, prostate, lung cancers, and for melanomas.

Each antigen represents a separate embodiment of the present invention.

The E7 protein that is utilized (either whole or as the source of the fragments) has, in another embodiment, the sequence.

MHGDTPTLHEYMLDLQPETTDLYCYEQLNDSSEEEDEIDGPAGQAEPD RAHYNIVTFCCKCDSTLRLCVQSTHVDIRTLEDLLMGTLGIVCPICSQKP (SEQ ID NO: 9). In another embodiment, the E7 protein is a homologue of SEQ ID NO: 9. In another embodiment, the E7 protein is a variant of SEQ ID NO: 9. In another embodiment, the E7 protein is an isomer of SEQ ID NO: 9. In another embodiment, the E7 protein is a fragment of SEQ ID NO: 9. In another embodiment, the E7 protein is a fragment of a homologue of SEQ ID NO: 9. In another embodiment, the E7 protein is a fragment of a variant of SEQ ID NO: 9. In another embodiment, the E7 protein is a fragment of an isomer of SEQ ID NO: 9. Each possibility represents a separate embodiment of the present invention.

In another embodiment, the sequence of the E7 protein is:

MHGPKATLQDIVLHLEPQNEIPVDLLCHEQLSDSEEENDEIDGVNHQH LPARRAEPQRHTMLCMCCKCEARIELVVESSADDLRAFQQLFLNTLSFVCPWCASQQ (SEQ ID NO: 10). In another embodiment, the E7 protein is a homologue of SEQ ID NO: 10. In another embodiment, the E7 protein is a variant of SEQ ID NO: 10. In another embodiment, the E7 protein is an isomer of SEQ ID NO: 10. In another embodiment, the E7 protein is a fragment of SEQ ID NO: 10. In another embodiment, the E7 protein is a fragment of a homologue of SEQ ID NO: 10. In another embodiment, the E7 protein is a fragment of a variant of SEQ ID NO: 10. In another embodiment, the E7 protein is a fragment of an isomer of SEQ ID NO: 10. Each possibility represents a separate embodiment of the present invention.

In another embodiment, the E7 protein has a sequence set forth in one of the following GenBank entries: M24214, NC_{—}004500, V01116, X62843, or M14119. In another embodiment, the E7 protein is a homologue of a sequence from one of the above GenBank entries. In another embodiment, the E7 protein is a variant of a sequence from one of the above GenBank entries. In another embodiment, the E7 protein is an isomer of a sequence from one of the above GenBank entries. In another embodiment, the E7 protein is a fragment of a sequence from one of the above GenBank entries. In another embodiment, the E7 protein is a fragment of a homologue of a sequence from one of the above GenBank entries. In another embodiment, the E7 protein is a fragment of a variant of a sequence from one of the above GenBank entries. In another embodiment, the E7 protein is a fragment of an isomer of a sequence from one of the above GenBank entries. Each possibility represents a separate embodiment of the present invention.

In one embodiment, the HPV16 E7 antigen is a peptide having the sequence: TLGIVCPI (SEQ ID NO: 11). In another embodiment, the HPV16 E7 antigen is a peptide having the sequence: LLMGTLGIV (SEQ ID NO: 12). In another embodiment, the HPV16 E7 antigen is a peptide having the sequence: YMLDLQPETT (SEQ ID NO: 13). In one embodiment, the HPV16 E7 antigen is a peptide comprising positions 86-93 of the wild-type HPV16 E7 antigen. In one embodiment, the HPV16 E7 antigen is a peptide comprising positions 82-90 of the wild-type HPV16 E7 antigen. In one embodiment, the HPV16 E7 antigen is a peptide comprising positions 11-20 of the wild-type HPV16 E7 antigen. In another embodiment, the HPV16 E7 antigen is a peptide consisting of positions 86-93, 82-90, or 11-20 of the wild-type HPV16 E7 antigen. In another embodiment, the HPV16 E7 antigen is a variant of a wild-type HPV16 E7 peptide. In another embodiment, the HPV16 E7 antigen is any HPV16 E7 antigen described in Ressing at al., J Immunol 1995 144 (11):5934-43, which is incorporated herein by reference in its entirety.

The NY-ESO-1 peptide of methods and compositions of the present invention is, in another embodiment, a peptide from an NY-ESO-1 protein, wherein the sequence of the protein is:

MQAEGRGTGGSTGDADGPGGPGIPDGPGGNAGGPGEAGATGGRGPRGAGA ARASGPGGGAPRGPHGGAASGLNGCCRCGARGPESRLLEFYLAMPFATPMEAELAR RSLAQDAPPLPVPGVLLKEFTVSGNILTIRLTAADHRQLQLSISSCLQQLSLLMWITQC FLPVFLAQPPSGQRR (SEQ ID NO: 14; GenBank Accession No. NM_{—}001327). In another embodiment, the NY-ESO-1 protein is a homologue of SEQ ID NO: 14. In another embodiment, the NY-ESO-1 protein is a variant of SEQ ID NO: 14. In another embodiment, the NY-ESO-1 protein is an isomer of SEQ ID NO: 14. In another embodiment, the NY-ESO-1 protein is a fragment of SEQ ID NO: 14. In another embodiment, the NY-ESO-1 protein is a fragment of a homologue of SEQ ID NO: 14. In another embodiment, the NY-ESO-1 protein is a fragment of a variant of SEQ ID NO: 14. In another embodiment, the NY-ESO-1 protein is a fragment of an isomer of SEQ ID NO: 14. Each possibility represents a separate embodiment of the present invention.

In another embodiment, the antigenic peptide of interest of methods and compositions of the present invention is an HPV E6 peptide. In another embodiment, the antigenic peptide is a whole E6 protein. In another embodiment, the antigenic peptide is a fragment of an E6 protein. Each possibility represents a separate embodiment of the present invention.

The E6 protein that is utilized (either whole or as the source of the fragments) has, in another embodiment, the sequence

MHQKRTAMFQDPQERPRKLPQLCTELQTTIHDIILECVYCKQQLLRREVYDF AFRDLCIVYRDGNPYAVCDKCLKFYSKISEYRHYCYSLYGTTLEQQYNKPLCDLLIRC INCQKPLCPEEKQRHLDKKQRFHNIRGRWTGRCMSCCRSSRTRRETQL (SEQ ID NO: 15). In another embodiment, the E6 protein is a homologue of SEQ ID NO: 15. In another embodiment, the E6 protein is a variant of SEQ ID NO: 15. In another embodiment, the E6 protein is an isomer of SEQ ID NO: 15. In another embodiment, the E6 protein is a fragment of SEQ ID NO: 15. In another embodiment, the E6 protein is a fragment of a homologue of SEQ ID NO: 15. In another embodiment, the E6 protein is a fragment of a variant of SEQ ID NO: 15. In another embodiment, the E6 protein is a fragment of an isomer of SEQ ID NO: 15. Each possibility represents a separate embodiment of the present invention.

In another embodiment, the sequence of the E6 protein is:

MARFEDPTRRPYKLPDLCTELNTSLQDIEITCVYCKTVLELTEVFEFAFKDLF VVYRDSIPHAACHKClDFYSRIRELRHYSDSVYGDTLEKLTNTGLYNLLIRCLRCQKP LNPAEKLRHLNEKRRFHNIAGHYRGQCHSCCNRARQERLQRRRETQV (SEQ ID NO: 16). In another embodiment, In another embodiment, the E6 protein is a homologue of SEQ ID NO: 16. In another embodiment, the E6 protein is a variant of SEQ ID NO: 16. In another embodiment, the E6 protein is an isomer of SEQ ID NO: 16. In another embodiment, the E6 protein is a fragment of SEQ ID NO: 16. In another embodiment, the E6 protein is a fragment of a homologue of SEQ ID NO: 16. In another embodiment, the E6 protein is a fragment of a variant of SEQ ID NO: 16. In another embodiment, the E6 protein is a fragment of an isomer of SEQ ID NO: 16. Each possibility represents a separate embodiment of the present invention.

In another embodiment, the E6 protein has a sequence set forth in one of the following GenBank entries: M24214, M14119, NC_{—}004500, V01116, X62843, or M14119. In another embodiment, the E6 protein is a homologue of a sequence from one of the above GenBank entries. In another embodiment, the E6 protein is a variant of a sequence from one of the above GenBank entries. In another embodiment, the E6 protein is an isomer of a sequence from one of the above GenBank entries. In another embodiment, the E6 protein is a fragment of a sequence from one of the above GenBank entries. In another embodiment, the E6 protein is a fragment of a homologue of a sequence from one of the above GenBank entries. In another embodiment, the E6 protein is a fragment of a variant of a sequence from one of the above GenBank entries. In another embodiment, the E6 protein is a fragment of an isomer of a sequence from one of the above GenBank entries. Each possibility represents a separate embodiment of the present invention.

The HPV that is the source of the heterologous antigen of methods of the present invention is, in another embodiment, an HPV 16. In another embodiment, the HPV is an HPV-18. In another embodiment, the HPV is selected from HPV-16 and HPV-18. In another embodiment, the HPV is an HPV-31. In another embodiment, the HPV is an HPV-35. In another embodiment, the HPV is an HPV-39. In another embodiment, the HPV is an HPV-45. In another embodiment, the HPV is an HPV-51. In another embodiment, the HPV is an HPV-52. In another embodiment, the HPV is an HPV-58. In another embodiment, the HPV is a high-risk HPV type. In another embodiment, the HPV is a mucosal HPV type. Each possibility represents a separate embodiment of the present invention.

In one embodiment, the Her2-neu chimeric protein, harbors two of the extracellular and one intracellular fragments of Her2/neu antigen showing clusters of MHC-class I epitopes of the oncogene, where, in another embodiment, the chimeric protein, harbors 3 H2Dq and at least 17 of the mapped human MHC-class I epitopes of the Her2/neu antigen. In another embodiment, the Her2-neu chimeric protein is fused to the first 441 amino acids of the Listeria-monocytogenes listeriolysin O (LLO) protein and expressed and secreted by the Listeria monocytogenes attenuated auxotrophic strain LmddA.

In another embodiment, the Her-2 chimeric protein is encoded by the following nucleic acid sequence set forth in SEQ ID NO:17

(SEQ ID NO: 17)
acccacctggacatgctccgccacctctaccagggctgccaggtggtgc
agggaaacctggaactcacctacctgcccaccaatgccagcctgtccac
ctgcaggatatccaggaggtgcagggctacgtgctcatcgctcacaacc
aagtgaggcaggtcccactgcagaggctgcggattgtgcgaggcaccca
gctctttgaggacaactatgccctggccgtgctagacaatggagacccg
ctgaacaataccacccctgtcacaggggcctccccaggaggcctgcggg
agctgcagcttcgaagcctcacagagatcttgaaaggaggggtcttgat
ccagcggaacccccagctctgctaccaggacacgattagtggaagaata
tccaggagtagctggctgcaagaagatctagggagcctggcatttctgc
cggagagctttgatggggacccagcctccaacactgccccgctccagcc
agagcagctccaagtgtttgagactctggaagagatcacaggttaccta
tacatctcagcatggccggacagcctgcctgacctcagcgtcttccaga
acctgcaagtaatccggggacgaattctgcacaatggcgcctactcgct
gaccctgcaagggctgggcatcagctggctggggctgcgctcactgagg
gaactgggcagtggactggccctcatccaccataacacccacctctgct
tcgtgcacacggtgccctgggaccagctctttcggaacccgcaccaagc
tctgctccacactgccaaccggccagaggacgagtgtgtgggcgagggc
ctggcctgccaccagctgtgcgcccgagggcagcagaagatccggaagt
acacgatgcggagactgctgcaggaaacggagctggtggagccgctgac
acctagcggagcgatgcccaaccaggcgcagatgcggatcctgaaagag
acggagctgaggaaggtgaaggtgcttggatctggcgcttttggcacag
tctacaagggcatctggatccctgatggggagaatgtgaaaattccagt
ggccatcaaagtgttgagggaaaacacatcccccaaagccaacaaagaa
atcttagacgaagcatacgtgatggctggtgtgggctccccatatgtct
cccgccactgggcatctgcctgacatccacggtgcagctggtgacacag
cttatgccctatggctgcctcttagactaa.

In another embodiment, the Her-2 chimeric protein has the sequence:

(SEQ ID NO: 18)
E T H L D M L R H L Y Q G C Q V V Q G N L E L T Y
L P T N A S L S F L Q D I Q E V Q G Y V L I A H N
Q V R Q V P L Q R L R I V R G T Q L F E D N Y A L
A V L D N G D P L N N T T P V T G A S P G G L R E
L Q L R S L T E I L K G G V L I Q R N P Q L C Y Q
D T I L W K N I Q E F A G C K K I F G S L A F L P
E S F D G D P A S N T A P L Q P E Q L Q V F E T L
E E I T G Y L Y I S A W P D S L P D L S V F Q N L
Q V I R G R I L H N G A Y S L T L Q G L G I S W L
G L R S L R E L G S G L A L I H H N T H L C F V H
T V P W D Q L F R N P H Q A L L H T A N R P E D E
C V G E G L A C H Q L C A R G Q Q K I R K Y T M R
R L L Q E T E L V E P L T P S G A M P N Q A Q M R
I L K E T E L R K V K V L G S G A F G T V Y K G I
W I P D G E N V K I P V A I K V L R E N T S P K A
N K E I L D E A Y V M A G V G S P Y V S R L L G I
C L T S T V Q L V T Q L M P Y G C L L D.

In one embodiment, the Her-2 chimeric protein or fragment thereof of the methods and compositions provided herein does not include a signal sequence thereof. In another embodiment, omission of the signal sequence enables the Her-2 fragment to be successfully expressed in Listeria, due the high hydrophobicity of the signal sequence.

In another embodiment, the fragment of a Her-2 chimeric protein of methods and compositions of the present invention does not include a transmembrane domain (TM) thereof. In one embodiment, omission of the TM enables the Her-2 fragment to be successfully expressed in Listeria, due the high hydrophobicity of the TM.

In one embodiment, the nucleic acid molecule provided herein is integrated into the Listeria genome. In another embodiment, the nucleic acid molecule is in a plasmid in the recombinant Listeria vaccine strain. In yet another embodiment, the plasmid is stably maintained in the recombinant Listeria vaccine strain in the absence of antibiotic selection. In another embodiment, the plasmid does not confer antibiotic resistance upon the recombinant Listeria.

In another embodiment, the term “nucleic acid” or grammatical equivalents herein refers to either DNA or RNA, or molecules which contain both ribo- and deoxyribonucleotides. The nucleic acids include genomic DNA, cDNA and oligonucleotides including sense and anti-sense nucleic acids. The nucleic acid may be double stranded, single stranded, or contain portions of both double stranded or single stranded sequence.

In one embodiment, the term “nucleic acid molecule” refers, in another embodiment, to a plasmid. In another embodiment, the term refers to an integration vector. In another embodiment, the term refers to a plasmid comprising an integration vector. In another embodiment, the integration vector is a site-specific integration vector. In another embodiment, a nucleic acid molecule of methods and compositions of the present invention are composed of any type of nucleotide known in the art. Each possibility represents a separate embodiment of the present invention.

In one embodiment the attenuated strain is LmddA. In another embodiment, this strain exerts a strong adjuvant effect which is an inherent property of Listeria-based vaccines. In another embodiment, Listeria-based vaccines reduce Tregs in breast cancer. In another embodiment, Listeria-based vaccines reduce Tregs in cancer, where in another embodiment is in breast. In another embodiment, the LmddA vector expressing HPV16 E7 is also associated with a significant decrease in the frequency of Tregs in the tumors.

In one embodiment, the terms “treating”, “therapeutic”, “therapy” are used interchangeably herein and refer to therapeutic treatment, while “inhibiting” and “suppressing” refer to prophylactic or preventative measures, wherein the object is to prevent or lessen the targeted pathologic condition or disorder as described herein. Thus, in one embodiment, treating may include directly affecting or curing the disease, disorder or condition and/or related symptoms, while suppressing or inhibiting may include preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “prophylaxis,” “prophylactic,” “preventing” or “inhibiting” refers, inter alia, to delaying the onset of symptoms, preventing relapse to a disease, decreasing the number or frequency of relapse episodes, increasing latency between symptomatic episodes, or a combination thereof. In one embodiment, “suppressing” refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. Each possibility represents a separate embodiment of the present invention.

In one embodiment, the nucleic acid molecule provided herein is used to transform the Listeria in order to arrive at a recombinant Listeria. In another embodiment, the recombinant Listeria is a Listeria monocytogenes (Lm) strain. In another embodiment, the nucleic acid provided herein used to transform Listeria lacks a virulence gene. In another embodiment, the nucleic acid molecule integrated into the Listeria genome carries a non-functional virulence gene. In another embodiment, the virulence gene is mutated in the recombinant Listeria. In yet another embodiment, the nucleic acid molecule is used to inactivate the endogenous gene present in the Listeria genome. In yet another embodiment, the virulence gene is an ActA gene or a PrfA gene. As will be understood by a skilled artisan, the virulence gene can be any gene known in the art to be associated with virulence in the recombinant Listeria.

In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. In another embodiment, the metabolic gene, virulence gene, etc. is lacking in the genome of the virulence strain. In one embodiment, the virulence gene is mutated in the chromosome. In another embodiment, the virulence gene is deleted from the chromosome. Each possibility represents a separate embodiment of the present invention.

In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. In another embodiment, the metabolic gene, virulence gene, etc. is lacking in the genome of the virulence strain. In one embodiment, the virulence gene is mutated in the chromosome. In another embodiment, the virulence gene is deleted from the chromosome. Each possibility represents a separate embodiment of the present invention.

In another embodiment, the nucleic acids and plasmids provided herein do not confer antibiotic resistance upon the recombinant Listeria.

“Stably maintained” refers, in another embodiment, to maintenance of a nucleic acid molecule or plasmid in the absence of selection (e.g. antibiotic selection) for 10 generations, without detectable loss. In another embodiment, the period is 14 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 140 generations. In another embodiment, the period is 200 generations. In another embodiment, the period is 300 generations. In another embodiment, the period is 500 generations. In another embodiment, the period is more than generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro. Each possibility represents a separate embodiment of the present invention.

In one embodiment, the present invention provides a recombinant Listeria strain expressing the antigen. The present invention also provides recombinant peptides comprising a listeriolysin (LLO) protein fragment fused to an antigen such as, but not limited to, a Her-2 chimeric protein or fragment thereof, vaccines and immunogenic compositions comprising same, and methods of inducing an anti-Her-2 immune response and treating and vaccinating against a Her-2-expressing tumor that has metastasized in the CNS, comprising the same.

In another embodiment, a recombinant Listeria strain of the present invention has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed by any other method known in the art.

In another embodiment, the polypeptide provided herein is a fusion protein comprising a non-hemolytic LLO protein or N-terminal fragment fused to a Her2/neu chimeric antigen. In another embodiment, a fusion protein of methods and compositions of the present invention comprises an ActA sequence from a Listeria organism fused to a Her2/neu chimeric antigen.

In one embodiment of the methods and compositions of the present invention, the fusion protein comprises a tumor antigen and an additional polypeptide. In another embodiment, the additional polypeptide is a non-hemolytic LLO protein or fragment thereof (Examples herein). In another embodiment, the additional polypeptide is a PEST sequence. In another embodiment, the additional polypeptide is an ActA protein or a fragment thereof. ActA proteins and fragments thereof augment antigen presentation and immunity in a similar fashion to LLO.

The additional polypeptide of methods and compositions of the present invention is, in another embodiment, a listeriolysin (LLO) peptide. In another embodiment, the additional polypeptide is an ActA peptide. In another embodiment, the additional polypeptide is a PEST sequence peptide. In another embodiment, the additional polypeptide is any other peptide capable of enhancing the immunogenicity of an antigen peptide. Each possibility represents a separate embodiment of the present invention.

Fusion proteins comprising a tumor antigen may be prepared by any suitable method, including, for example, cloning and restriction of appropriate sequences or direct chemical synthesis by methods discussed below. Alternatively, subsequences may be cloned and the appropriate subsequences cleaved using appropriate restriction enzymes. The fragments may then be ligated to produce the desired DNA sequence. In one embodiment, DNA encoding the antigen can be produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid. Each method represents a separate embodiment of the present invention.

In another embodiment of methods and compositions of the present invention, a polypeptide encoded by a nucleic acid sequence of methods and compositions of the present invention is a fusion protein comprising a tumor antigen and an additional polypeptide, where in another embodiment, the fusion protein comprises, inter alia, an Lm non-hemolytic LLO protein (Examples herein).

In another embodiment, provided herein is a vaccine comprising a recombinant polypeptide of the present invention.

In another embodiment, provided herein is a nucleotide molecule encoding a recombinant polypeptide of the present invention. In another embodiment, provided herein is a vaccine comprising the nucleotide molecule.

In another embodiment, provided herein is a nucleotide molecule encoding a recombinant polypeptide of the present invention.

In another embodiment, provided herein is a recombinant polypeptide encoded by the nucleotide molecule of the present invention.

In another embodiment, provided herein is a vaccine comprising a nucleotide molecule or recombinant polypeptide of the present invention.

In another embodiment, provided herein is an immunogenic composition comprising a nucleotide molecule or recombinant polypeptide of the present invention.

In another embodiment, provided herein is a vector comprising a nucleotide molecule or recombinant polypeptide of the present invention.

In another embodiment, provided herein is a recombinant form of Listeria comprising a nucleotide molecule of the present invention.

In another embodiment, provided herein is a vaccine comprising a recombinant form of Listeria of the present invention.

In another embodiment, provided herein is a culture of a recombinant form of Listeria of the present invention.

In another embodiment, the Listeria of methods and compositions of the present invention is Listeria monocytogenes. In another embodiment, the Listeria is Listeria ivanovii. In another embodiment, the Listeria is Listeria welshimeri. In another embodiment, the Listeria is Listeria seeligeri. Each type of Listeria represents a separate embodiment of the present invention.

In one embodiment, the vaccine for use in the methods of the present invention comprises a recombinant Listeria monocytogenes, in any form or embodiment as described herein. In one embodiment, the vaccine for use in the present invention consists of a recombinant Listeria monocytogenes of the present invention, in any form or embodiment as described herein. In another embodiment, the vaccine for use in the methods of the present invention consists essentially of a recombinant Listeria monocytogenes of the present invention, in any form or embodiment as described herein. In one embodiment, the term “comprise” refers to the inclusion of a recombinant Listeria monocytogenes in the vaccine, as well as inclusion of other vaccines or treatments that may be known in the art. In another embodiment, the term “consisting essentially of” refers to a vaccine, whose functional component is the recombinant Listeria monocytogenes, however, other components of the vaccine may be included that are not involved directly in the therapeutic effect of the vaccine and may, for example, refer to components which facilitate the effect of the recombinant Listeria monocytogenes (e.g. stabilizing, preserving, etc.). In another embodiment, the term “consisting” refers to a vaccine, which contains the recombinant Listeria monocytogenes.

In another embodiment, the methods of the present invention comprise the step of administering a recombinant Listeria monocytogenes, in any form or embodiment as described herein. In one embodiment, the methods of the present invention consist of the step of administering a recombinant Listeria monocytogenes of the present invention, in any form or embodiment as described herein. In another embodiment, the methods of the present invention consist essentially of the step of administering a recombinant Listeria monocytogenes of the present invention, in any form or embodiment as described herein. In one embodiment, the term “comprise” refers to the inclusion of the step of administering a recombinant Listeria monocytogenes in the methods, as well as inclusion of other methods or treatments that may be known in the art. In another embodiment, the term “consisting essentially of” refers to a methods, whose functional component is the administration of recombinant Listeria monocytogenes, however, other steps of the methods may be included that are not involved directly in the therapeutic effect of the methods and may, for example, refer to steps which facilitate the effect of the administration of recombinant Listeria monocytogenes. In one embodiment, the term “consisting” refers to a method of administering recombinant Listeria monocytogenes with no additional steps.

In one embodiment, the Listeria strain of the methods and compositions of the present invention is the ADXS31-164 strain. In another embodiment, ADXS31-164 stimulates the secretion of IFN-γ by the splenocytes from wild type FVB/N mice. Further, the data presented herein show that ADXS31-164 is able to elicit anti-Her2/neu specific immune responses to human epitopes that are located at different domains of the targeted antigen.

In another embodiment, the Listeria strain of the methods and compositions of the present invention is the LmddA174, LmddA265, LmddA266, LmddA174, LmddA257, or the LmddA256 strain (see Example 1).

In another embodiment, the present invention provides a recombinant form of Listeria comprising a nucleotide molecule encoding a tumor antigen provided herein or a functional fragment thereof.

In one embodiment, the fusion protein of methods and compositions of the present invention comprises an LLO signal sequence from LLO. In another embodiment, the two molecules of the fusion protein are joined directly. In another embodiment, the two molecules are joined by a short spacer peptide, consisting of one or more amino acids. In one embodiment, the spacer has no specific biological activity other than to join the proteins or to preserve some minimum distance or other spatial relationship between them. In another embodiment, the constituent amino acids of the spacer are selected to influence some property of the molecule such as the folding, net charge, or hydrophobicity. In another embodiment, the two molecules of the protein (the LLO fragment and the antigen) are synthesized separately or unfused. In another embodiment, the two molecules of the protein are synthesized separately from the same nucleic acid. In yet another embodiment, the two molecules are individually synthesized from separate nucleic acids. Each possibility represents a separate embodiment of the present invention.

Point mutations or amino-acid deletions in the oncogenic protein Her2/neu, have been reported to mediate treatment of resistant tumor cells, when these tumors have been targeted by small fragment Listeria-based vaccines or trastuzumab (a monoclonal antibody against an epitope located at the extracellular domain of the Her2/neu antigen). Described herein is a chimeric Her2/neu based composition which harbors two of the extracellular and one intracellular fragments of Her2/neu antigen showing clusters of MHC-class I epitopes of the oncogene. This chimeric protein, which harbors 3 H2Dq and at least 17 of the mapped human MHC-class I epitopes of the Her2/neu antigen was fused to the first 441 amino acids of the Listeria-monocytogenes listeriolysin O protein and expressed and secreted by the Listeria monocytogenes attenuated strain LmddA.

In one embodiment, the methods and compositions provided herein further comprise an adjuvant, where in another embodiment, the adjuvant comprises a granulocyte/macrophage colony-stimulating factor (GM-CSF) protein, a nucleotide molecule encoding a GM-CSF protein, saponin QS21, monophosphoryl lipid A, or an unmethylated CpG-containing oligonucleotide.

In one embodiment, attenuated Listeria strains, such as Lm delta-actA mutant (Brundage et al, 1993, Proc. Natl. Acad. Sci., USA, 90:11890-11894), L. monocytogenes delta-plcA (Camilli et al, 1991, J. Exp. Med., 173:751-754), or delta-ActA, delta INL-b (Brockstedt et 5 al, 2004, PNAS, 101:13832-13837) are used in the present invention. In another embodiment, attenuated Listeria strains are constructed by introducing one or more attenuating mutations, as will be understood by one of average skill in the art when equipped with the disclosure herein. Examples of such strains include, but are not limited to Listeria strains auxotrophic for aromatic amino acids (Alexander et al, 1993, Infection and Immunity 10 61:2245-2248) and mutant for the formation of lipoteichoic acids (Abachin et al, 2002, Mol. Microbiol. 43:1-14) and those attenuated by a lack of a virulence gene (see examples herein).

In another embodiment, the nucleic acid molecule of methods and compositions of the present invention is operably linked to a promoter/regulatory sequence. In another embodiment, the first open reading frame of methods and compositions of the present invention is operably linked to a promoter/regulatory sequence. In another embodiment, the second open reading frame of methods and compositions of the present invention is operably linked to a promoter/regulatory sequence. In another embodiment, each of the open reading frames are operably linked to a promoter/regulatory sequence. Each possibility represents a separate embodiment of the present invention.

The skilled artisan, when equipped with the present disclosure and the methods provided herein, will readily understand that different transcriptional promoters, terminators, carrier vectors or specific gene sequences (e.g. those in commercially available cloning vectors) can be used successfully in methods and compositions of the present invention. As is contemplated in the present invention, these functionalities are provided in, for example, the commercially available vectors known as the pUC series. In another embodiment, non-essential DNA sequences (e.g. antibiotic resistance genes) are removed. Each possibility represents a separate embodiment of the present invention. In another embodiment, a commercially available plasmid is used in the present invention. Such plasmids are available from a variety of sources, for example, Invitrogen (La Jolla, Calif.), Stratagene (La Jolla, Calif.), Clontech (Palo Alto, Calif.), or can be constructed using methods well known in the art.

In one embodiment, extraneous nucleotide sequences are removed to decrease the size of a plasmid used to express the nucleotide molecule of the present invention and increase the size of the cassette that can be placed therein.

Such methods are well known in the art, and are described in, for example, Sambrook et al. (1989, Molecular Cloning: A Laboratory Manual, Cold Spring Harbor Laboratory Press, New York) and Ausubei et al. (1997, Current Protocols in Molecular Biology, Green & Wiley, New York).

In one embodiment, antibiotic resistance genes are used in the conventional selection and cloning processes commonly employed in molecular biology and vaccine preparation. Antibiotic resistance genes contemplated in the present invention include, but are not limited to, gene products that confer resistance to ampicillin, penicillin, methicillin, streptomycin, erythromycin, kanamycin, tetracycline, cloramphenicol (CAT), neomycin, hygromycin, gentamicin and others well known in the art. Each gene represents a separate embodiment of the present invention.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, D.C.; Miller, 1992, A Short Course in Bacterial Genetics, Cold Spring Harbor Laboratory Press, Cold Spring Harbor, N.Y.) In another embodiment, the Listeria vaccine strain of the present invention is transformed by electroporation. Each method represents a separate embodiment of the present invention.

In another embodiment, conjugation is used to introduce genetic material and/or plasmids into bacteria. Methods for conjugation are well known in the art, and are described, for example, in Nikodinovic J et al. (A second generation snp-derived Escherichia coli-Streptomyces shuttle expression vector that is generally transferable by conjugation. Plasmid. 2006 November; 56 (3):223-7) and Auchtung J M et al (Regulation of a Bacillus subtilis mobile genetic element by intercellular signaling and the global DNA damage response. Proc Natl Acad Sci USA. 2005 Aug. 30; 102 (35):12554-9). Each method represents a separate embodiment of the present invention.

“Transforming,” in one embodiment, is used identically with the term “transfecting,” and refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the present invention.

Plasmids and other expression vectors useful in the present invention can include features such as a promoter/regulatory sequence, an origin of replication for gram negative and gram positive bacteria, an isolated nucleic acid encoding a fusion protein and an isolated nucleic acid encoding an amino acid metabolism gene. Further, an isolated nucleic acid encoding a fusion protein and an amino acid metabolism gene will have a promoter suitable for driving expression of such an isolated nucleic acid. Promoters useful for driving expression in a bacterial system are well known in the art, and include bacteriophage lambda, the bla promoter of the beta-lactamase gene of pBR322, and the CAT promoter of the chloramphenicol acetyl transferase gene of pBR325. Further examples of prokaryotic promoters include the major right and left promoters of 5 bacteriophage lambda (PL and PR), the trp, recA, lacZ, lad, and gal promoters of E. coli, the alpha-amylase (Ulmanen et al, 1985. J. Bacteriol. 162:176-182) and the S28-specific promoters of B. subtilis (Gilman et al, 1984 Gene 32:11-20), the promoters of the bacteriophages of Bacillus (Gryczan, 1982, In: The Molecular Biology of the Bacilli, Academic Press, Inc., New York), and Streptomyces promoters (Ward et al, 1986, Mol. Gen. Genet. 203:468-478). Additional prokaryotic promoters contemplated in the present invention are reviewed in, for example, Glick (1987, J. Ind. Microbiol. 1:277-282); Cenatiempo, (1986, Biochimie, 68:505-516); and Gottesman, (1984, Ann Rev. Genet. 18:414-442). Further examples of promoter/regulatory elements contemplated in the present invention include, but are not limited to the Listerial prfA promoter, the Listerial hly promoter, the Listerial p60 promoter and the Listerial ActA promoter (GenBank Acc. No. NC_{—}003210) or fragments thereof.

In another embodiment, a plasmid of methods and compositions of the present invention comprises a gene encoding a fusion protein. In another embodiment, subsequences are cloned and the appropriate subsequences cleaved using appropriate restriction enzymes. The fragments are then, in another embodiment, ligated to produce the desired DNA sequence. In another embodiment, DNA encoding the antigen is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid. Each method represents a separate embodiment of the present invention.

In one embodiment, the present invention further comprises a phage based chromosomal integration system for clinical applications. A host strain that is auxotrophic for essential enzymes, including, but not limited to, d-alanine racemase will be used, for example Lmdal(−)dat(−). In another embodiment, in order to avoid a “phage curing step,” a phage integration system based on PSA is used (Lauer, et al., 2002 J Bacteriol, 184:4177-4186). This requires, in another embodiment, continuous selection by antibiotics to maintain the integrated gene. Thus, in another embodiment, the current invention enables the establishment of a phage based chromosomal integration system that does not require selection with antibiotics. Instead, an auxotrophic host strain will be complemented.

The recombinant proteins of the present invention are synthesized, in another embodiment, using recombinant DNA methodology. This involves, in one embodiment, creating a DNA sequence that encodes the fusion protein, placing the DNA in an expression cassette, such as the plasmid of the present invention, under the control of a particular promoter/regulatory element, and expressing the protein. DNA encoding the fusion protein (e.g. non-hemolytic LLO/antigen) of the present invention is prepared, in another embodiment, by any suitable method, including, for example, cloning and restriction of appropriate sequences or direct chemical synthesis by methods such as the phosphotriester method of Narang et al. (1979, Meth. Enzymol. 68: 90-99); the phosphodiester method of Brown et al. (1979, Meth. Enzymol 68: 109-141); the diethylphosphoramidite method of Beaucage et al. (1981, Tetra. Lett., 22: 14 1859-1862); and the solid support method of U.S. Pat. No. 4,458,066.

In another embodiment, chemical synthesis is used to produce a single stranded oligonucleotide. This single stranded oligonucleotide is converted, in various embodiments, into double stranded DNA by hybridization with a complementary sequence, or by polymerization with a DNA polymerase using the single strand as a template. One of skill in the art would recognize that while chemical synthesis of DNA is limited to sequences of about 100 bases, longer sequences can be obtained by the ligation of shorter sequences. In another embodiment, subsequences are cloned and the appropriate subsequences cleaved using appropriate restriction enzymes. The fragments are then be ligated to produce the desired DNA sequence.

In another embodiment, DNA encoding the fusion protein or the recombinant protein of the present invention is cloned using DNA amplification methods such as polymerase chain reaction (PCR). Thus, the gene for non-hemolytic LLO is PCR amplified, using a sense primer comprising a suitable restriction site and an antisense primer comprising another restriction site, e.g. a non-identical restriction site to facilitate cloning. The same is repeated for the isolated nucleic acid encoding an antigen. Ligation of the non-hemolytic LLO and antigen sequences and insertion into a plasmid or vector produces a vector encoding non-hemolytic LLO joined to a terminus of the antigen. The two molecules are joined either directly or by a short spacer introduced by the restriction site.

In another embodiment, the molecules are separated by a peptide spacer consisting of one or more amino acids, generally the spacer will have no specific biological activity other than to join the proteins or to preserve some minimum distance or other spatial relationship between them. In another embodiment, the constituent AA of the spacer are selected to influence some property of the molecule such as the folding, net charge, or hydrophobicity. In another embodiment, the nucleic acid sequences encoding the fusion or recombinant proteins are transformed into a variety of host cells, including E. coli, other bacterial hosts, such as Listeria, yeast, and various higher eukaryotic cells such as the COS, CHO and HeLa cells lines and myeloma cell lines. The recombinant fusion protein gene will be operably linked to appropriate expression control sequences for each host. Promoter/regulatory sequences are described in detail elsewhere herein. In another embodiment, the plasmid further comprises additional promoter regulatory elements, as well as a ribosome binding site and a transcription termination signal. For eukaryotic cells, the control sequences will include a promoter and an enhancer derived from e.g. immunoglobulin genes, SV40, cytomegalovirus, etc., and a polyadenylation sequence. In another embodiment, the sequences include splice donor and acceptor sequences.

In one embodiment, the term “operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences.

In another embodiment, in order to select for an auxotrophic bacteria comprising the plasmid, transformed auxotrophic bacteria are grown on a media that will select for expression of the amino acid metabolism gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for Dglutamic acid synthesis, will not grow. In another embodiment, a bacterium auxotrophic for D-alanine synthesis will grow in the absence of D-alanine when transformed and expressing the plasmid of the present invention if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.). Each method represents a separate embodiment of the present invention.

In another embodiment, once the auxotrophic bacteria comprising the plasmid of the present invention have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria vaccine vector by adjusting the volume of the media in which the auxotrophic bacteria comprising the plasmid are growing.

The skilled artisan will appreciate that, in another embodiment, other auxotroph strains and complementation systems are adopted for the use with this invention.

In another embodiment, provided herein is a method of eliciting an enhanced immune response to a tumor in the CNS of a subject, whereby and in another embodiment, the method comprises the step of administering to the subject a composition comprising the recombinant Listeria vaccine strain described herein. In yet another embodiment, the immune response against the primary tumor comprises an immune response to at least one subdominant epitope of the tumor.

In another embodiment, provided herein is a method of preventing the onset of a tumor in the CNS of a subject, whereby and in another embodiment, the method comprises the step of administering to the subject a composition comprising the recombinant Listeria vaccine strain provided herein.

In one embodiment, provided herein is a method of decreasing the frequency of intra-tumoral T regulatory cells in the CNS of a subject, whereby and in another embodiment, the method comprises the step of administering to the subject a composition comprising the recombinant Listeria vaccine strain provided herein.

In another embodiment, provided herein is a method of decreasing the frequency of intra-tumoral myeloid derived suppressor cells in the CNS of a subject, whereby and in another embodiment, the method comprises the step of administering to the subject a composition comprising the recombinant Listeria vaccine strain provided herein.

In one embodiment, provided herein a method of preventing the formation of a tumor in the CNS of a subject, whereby and in another embodiment, the method comprises the step of administering to the subject a composition comprising the recombinant Listeria vaccine strain provided herein.

In one embodiment, the recombinant Listeria vaccine strain is peripherally administered to the subject. In another embodiment, the recombinant live Listeria is administered through means known in the art, including but not limited to being administered intravenously, intranasally, or intramuscularly. In another embodiment, the recombinant live Listeria is administered through means known in the art, including but not limited to being administered intravenously, intranasally, or intramuscularly.

In another embodiment, provided herein is a method of treating a tumor in the CNS of a subject, whereby and in another embodiment, the method comprises the step of administering to the subject a composition comprising the recombinant Listeria vaccine strain provided herein.

In one embodiment, provided herein is a method of administering the composition of the present invention. In another embodiment, provided herein is a method of administering the vaccine of the present invention. In another embodiment, provided herein is a method of administering the recombinant polypeptide or recombinant nucleotide of the present invention. In another embodiment, the step of administering the composition, vaccine, recombinant polypeptide or recombinant nucleotide of the present invention is performed with an attenuated recombinant form of Listeria comprising the composition, vaccine, recombinant nucleotide or expressing the recombinant polypeptide, each in its own discrete embodiment. In another embodiment, the administering is performed with a different attenuated bacterial vector. In another embodiment, the administering is performed with a different attenuated Listeria vector. In another embodiment, the administering is performed with a different attenuated Listeria monocytogenes vector.

In another embodiment, the immune response elicited by recombinant Lm-based vaccine provided herein comprises a CD8⁺ T cell-mediated response that effects a therapeutic response across the blood brain barrier that treats a CNS cancer. In another embodiment, the immune response consists primarily of a CD8⁺ T cell-mediated response. In another embodiment, the only detectable component of the immune response is a CD8⁺ T cell-mediated response.

Immunization with Lm has been shown to result in the generation of “high quality” effector CD4⁺ T cells capable of secreting multiple cytokines such as IFN-γ and TNF-α, or three cytokines such as TNF-α, IFN-γ and IL-2, coincident with the generation of a memory CD4⁺ T cell response. In another embodiment, the immune response elicited by recombinant Lm-based vaccine provided herein comprises a CD4⁺ T cell-mediated response that effects a therapeutic response across the blood brain barrier that treats a CNS cancer. In another embodiment, the immune response consists primarily of a CD4⁺ T cell-mediated response. In another embodiment, the only detectable component of the immune response is a CD4⁺ T cell-mediated response. In another embodiment, the CD4⁺ T cell-mediated response is accompanied by a measurable antibody response against the antigen. In another embodiment, the CD4⁺ T cell-mediated response is not accompanied by a measurable antibody response against the antigen.

In another embodiment, provided herein a method of inducing a CD8⁺ T cell-mediated immune response in the CNS of a subject against a subdominant CD8⁺ T cell epitope of an antigen, comprising the steps of (a) fusing a nucleotide molecule encoding the Her2-neu chimeric antigen or a fragment thereof to a nucleotide molecule encoding an N-terminal fragment of a LLO protein, thereby creating a recombinant nucleotide encoding an LLO-antigen fusion protein; and (b) administering the recombinant nucleotide or the LLO-antigen fusion to the subject; thereby inducing a CD8⁺ T cell-mediated immune response against a subdominant CD8⁺ T cell epitope of an antigen.

In one embodiment, expanded intracerebral immunity against endogenous tumor-associated antigens is dependent on both CD4⁺ and CD8⁺ T cells.

In another embodiment, the immune response elicited by recombinant Lm-based vaccine provided herein comprises an immune response to at least one subdominant epitope of the antigen. In another embodiment, the immune response consists primarily of an immune response to at least one subdominant epitope. In another embodiment, the only measurable component of the immune response is an immune response to at least one subdominant epitope. Each type of immune response represents a separate embodiment of the present invention.

In another embodiment, the dominant epitope or subdominant epitope is dominant or subdominant, respectively, in the subject being treated. In another embodiment, the dominant epitope or subdominant epitope is dominant or subdominant in a population being treated.

In one embodiment, the vaccines of the present invention comprise an adjuvant, while in another embodiment, the vaccines do not comprise an adjuvant. “Adjuvant” refers, in another embodiment, to compounds that, when administered to an individual or tested in vitro, increase the immune response to an antigen in the individual or test system to which the antigen is administered. In another embodiment, an immune adjuvant enhances an immune response to an antigen that is weakly immunogenic when administered alone, i.e., inducing no or weak antibody titers or cell-mediated immune response. In another embodiment, the adjuvant increases antibody titers to the antigen. In another embodiment, the adjuvant lowers the dose of the antigen effective to achieve an immune response in the individual.

The adjuvant utilized in methods and compositions of the present invention is, in another embodiment, a CpG-containing nucleotide sequence. In another embodiment, the adjuvant is a CpG-containing oligonucleotide. In another embodiment, the adjuvant is a CpG-containing oligodeoxynucleotide (CpG ODN). In another embodiment, the adjuvant is ODN 1826. In another embodiment, the adjuvant is an aluminum salt adjuvant. In another embodiment, the aluminum salt adjuvant is an alum-precipitated vaccine. In another embodiment, the aluminum salt adjuvant is an alum-adsorbed vaccine. Aluminum-salt adjuvants are well known in the art and are described, for example, in Harlow, E. and D. Lane (1988; Antibodies: A Laboratory Manual Cold Spring Harbor Laboratory) and Nicklas, W. (1992; Aluminum salts. Research in Immunology 143:489-493).

In another embodiment, the adjuvant is a Montanide ISA adjuvant. In another embodiment, the adjuvant is a trimer of complement component C3d. In another embodiment, the trimer is covalently linked to the protein immunogen. In another embodiment, the adjuvant is MF59. In another embodiment, the adjuvant is a granulocyte/macrophage colony-stimulating factor (GM-CSF) protein. In another embodiment, the adjuvant is a mixture comprising a GM-CSF protein. In another embodiment, the adjuvant is a nucleotide molecule encoding GM-CSF. In another embodiment, the adjuvant is a mixture comprising a nucleotide molecule encoding GM-CSF. In another embodiment, the adjuvant is saponin QS21. In another embodiment, the adjuvant is a mixture comprising saponin QS21. In another embodiment, the adjuvant is monophosphoryl lipid A (MPL). In another embodiment, the adjuvant is a mixture comprising MPL. In another embodiment, the adjuvant is SBAS2. In another embodiment, the adjuvant is a mixture comprising SBAS2. In another embodiment, the adjuvant is an unmethylated CpG-containing oligonucleotide. In another embodiment, the adjuvant is a mixture comprising an unmethylated CpG-containing oligonucleotide. In another embodiment, the adjuvant is an immune-stimulating cytokine. In another embodiment, the adjuvant is a mixture comprising an immune-stimulating cytokine. In another embodiment, the adjuvant is a nucleotide molecule encoding an immune-stimulating cytokine. In another embodiment, the adjuvant is a mixture comprising a nucleotide molecule encoding an immune-stimulating cytokine. In another embodiment, the adjuvant is a mixture comprising a quill glycoside. In another embodiment, the adjuvant is a mixture comprising a bacterial mitogen. In another embodiment, the adjuvant is a mixture comprising a bacterial toxin. In another embodiment, the adjuvant is a mixture comprising any other adjuvant known in the art. In another embodiment, the adjuvant is a mixture of 2 of the above adjuvants. In another embodiment, the adjuvant is a mixture of 3 of the above adjuvants. In another embodiment, the adjuvant is a mixture of more than three of the above adjuvants.

In another embodiment, the methods of the present invention further comprises the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost vaccine is experimentally determined by the skilled artisan. In another embodiment, the period between a prime and a boost vaccine is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost vaccine is administered 8-10 weeks after the prime vaccine.

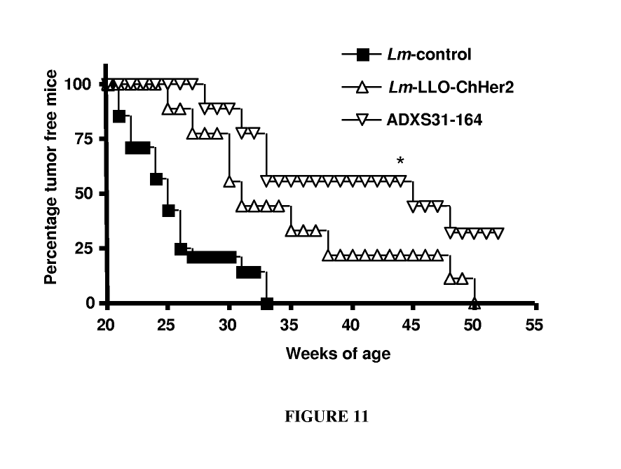

In another embodiment, the immune response elicited by recombinant Lm-based vaccine provided herein comprises a cytokine and chemokine immune response that effects a therapeutic response across the blood brain barrier that treats a CNS cancer. In another embodiment, the CNS cancer is Glioblastoma multiforme (GBM). In another embodiment, the present invention provides the surprising result that autochthonous tumor formation in transgenic animals is prevented for an unexpectedly prolonged period (see Example 4, and FIG. 11) and this proves the efficiency for a glioma vaccine, as provided herein, since the majority of GBM are believed to be Her2 positive.

In another embodiment, the methods of the present invention further comprises the step of administering to the subject a booster vaccination. In one embodiment, the booster vaccination follows a single priming vaccination. In another embodiment, a single booster vaccination is administered after the priming vaccinations. In another embodiment, two booster vaccinations are administered after the priming vaccinations. In another embodiment, three booster vaccinations are administered after the priming vaccinations. In one embodiment, the period between a prime and a boost vaccine is experimentally determined by the skilled artisan. In another embodiment, the period between a prime and a boost vaccine is 1 week, in another embodiment it is 2 weeks, in another embodiment, it is 3 weeks, in another embodiment, it is 4 weeks, in another embodiment, it is 5 weeks, in another embodiment it is 6-8 weeks, in yet another embodiment, the boost vaccine is administered 8-10 weeks after the prime vaccine.

In another embodiment, the booster vaccination comprises the use of an alternate form of a vaccine different to that of the priming vaccine. In another embodiment, the different or alternate form of the vaccine is a DNA vaccine encoding the fusion protein, a recombinant polypeptide comprising said fusion protein or a live recombinant Listeria vaccine vector.

Methods of measuring immune responses are well known in the art, and include, e.g. measuring suppression of tumor growth, flow cytometry, target cell lysis assays (e.g. chromium release assay), the use of tetramers, and others. Each method represents a separate embodiment of the present invention.

In one embodiment, the present invention provides a method for “epitope spreading” of a tumor. In another embodiment, the immunization using the compositions and methods provided herein induce epitope spreading onto other tumors bearing antigens other than the antigen carried in the vaccine of the present invention.

In one embodiment, provided herein is a method of treating, suppressing, or inhibiting a cancer or a tumor growth in a subject by epitope spreading whereby and in another embodiment, said cancer is associated with expression of an antigen or fragment thereof comprised in the composition of the present invention. In another embodiment, the method comprises administering to said subject a composition comprising the recombinant polypeptide, recombinant Listeria, or recombinant vector of the present invention. In yet another embodiment, the subject mounts an immune response against the antigen-expressing cancer or the antigen-expressing tumor, thereby treating, suppressing, or inhibiting a cancer or a tumor growth in a subject.

“Dominant CD8⁺ T cell epitope,” in one embodiment, refers to an epitope that is recognized by over 30% of the antigen-specific CD8⁺ T cells that are elicited by vaccination, infection, or a malignant growth with a protein or a pathogen or cancer cell containing the protein. In another embodiment, the term refers to an epitope recognized by over 35% of the antigen-specific CD8⁺ T cells that are elicited thereby. In another embodiment, the term refers to an epitope recognized by over 40% of the antigen-specific CD8⁺ T cells. In another embodiment, the term refers to an epitope recognized by over 45% of the antigen-specific CD8⁺ T cells. In another embodiment, the term refers to an epitope recognized by over 50% of the antigen-specific CD8⁺ T cells. In another embodiment, the term refers to an epitope recognized by over 55% of the antigen-specific CD8⁺ T cells. In another embodiment, the term refers to an epitope recognized by over 60% of the antigen-specific CD8⁺ T cells. In another embodiment, the term refers to an epitope recognized by over 65% of the antigen-specific CD8⁺ T cells. In another embodiment, the term refers to an epitope recognized by over 70% of the antigen-specific CD8⁺ T cells. In another embodiment, the term refers to an epitope recognized by over 75% of the antigen-specific CD8⁺ T cells. In another embodiment, the term refers to an epitope recognized by over 80% of the antigen-specific CD8⁺ T cells. In another embodiment, the term refers to an epitope recognized by over 85% of the antigen-specific CD8⁺ T cells. In another embodiment, the term refers to an epitope recognized by over 90% of the antigen-specific CD8⁺ T cells. In another embodiment, the term refers to an epitope recognized by over 95% of the antigen-specific CD8⁺ T cells. In another embodiment, the term refers to an epitope recognized by over 96% of the antigen-specific CD8⁺ T cells. In another embodiment, the term refers to an epitope recognized by over 97% of the antigen-specific CD8⁺ T cells. In another embodiment, the term refers to an epitope recognized by over 98% of the antigen-specific CD8⁺ T cells.

“Subdominant CD8⁺ T cell epitope”, in one embodiment, refers to an epitope recognized by fewer than 30% of the antigen-specific CD8⁺ T cells that are elicited by vaccination, infection, or a malignant growth with a protein or a pathogen or cancer cell containing the protein. In another embodiment, the term refers to an epitope recognized by fewer than 28% of the antigen-specific CD8⁺ T cells. In another embodiment, the term refers to an epitope recognized by over 26% of the antigen-specific CD8⁺ T cells. In another embodiment, the term refers to an epitope recognized by fewer than 24% of the antigen-specific CD8⁺ T cells. In another embodiment, the term refers to an epitope recognized by over 22% of the antigen-specific CD8⁺ T cells. In another embodiment, the term refers to an epitope recognized by fewer than 20% of the antigen-specific CD8⁺ T cells. In another embodiment, the term refers to an epitope recognized by over 18% of the antigen-specific CD8⁺ T cells. In another embodiment, the term refers to an epitope recognized by fewer than 16% of the antigen-specific CD8⁺ T cells. In another embodiment, the term refers to an epitope recognized by over 14% of the antigen-specific CD8⁺ T cells. In another embodiment, the term refers to an epitope recognized by over 12% of the antigen-specific CD8⁺ T cells. In another embodiment, the term refers to an epitope recognized by fewer than 10% of the antigen-specific CD8⁺ T cells. In another embodiment, the term refers to an epitope recognized by over 8% of the antigen-specific CD8⁺ T cells. In another embodiment, the term refers to an epitope recognized by fewer than 6% of the antigen-specific CD8⁺ T cells. In another embodiment, the term refers to an epitope recognized by fewer than 5% of the antigen-specific CD8⁺ T cells. In another embodiment, the term refers to an epitope recognized by over 4% of the antigen-specific CD8⁺ T cells. In another embodiment, the term refers to an epitope recognized by fewer than 3% of the antigen-specific CD8⁺ T cells. In another embodiment, the term refers to an epitope recognized by fewer than 2% of the antigen-specific CD8⁺ T cells. In another embodiment, the term refers to an epitope recognized by fewer than 1% of the antigen-specific CD8⁺ T cells. In another embodiment, the term refers to an epitope recognized by fewer than 0.5% of the antigen-specific CD8⁺ T cells.

Each type of the dominant epitope and subdominant epitope represents a separate embodiment of the present invention.

The antigen in methods and compositions of the present invention is, in one embodiment, expressed at a detectable level on a non-tumor cell of the subject. In another embodiment, the antigen is expressed at a detectable level on at least a certain percentage (e.g. 0.01%, 0.03%, 0.1%, 0.3%, 1%, 2%, 3%, or 5%) of non-tumor cells of the subject. In one embodiment, “non-tumor cell” refers to a cell outside the body of the tumor. In another embodiment, “non-tumor cell” refers to a non-malignant cell. In another embodiment, “non-tumor cell” refers to a non-transformed cell. In another embodiment, the non-tumor cell is a somatic cell. In another embodiment, the non-tumor cell is a germ cell. Each possibility represents a separate embodiment of the present invention.

“Detectable level” refers, in one embodiment, to a level detectable by a standard assay. In one embodiment, the assay is an immunological assay. In one embodiment, the assay is enzyme-linked immunoassay (ELISA). In another embodiment, the assay is Western blot. In another embodiment, the assay is FACS. It is to be understood by a skilled artisan that any other assay available in the art can be used in the methods provided herein. In another embodiment, a detectable level is determined relative to the background level of a particular assay. Methods for performing each of these techniques are well known to those skilled in the art, and each technique represents a separate embodiment of the present invention.

In one embodiment, vaccination with recombinant antigen-expressing Lm induces epitope spreading. In another embodiment, vaccination with LLO-antigen fusions, ActA-antigen fusions, or PEST-antigen fusions, induces epitope spreading as well. Each possibility represents a separate embodiment of the present invention.

In another embodiment, the present invention provides a method for inducing formation of cytotoxic T cells in a host having a CNS cancer, comprising administering to the host a composition of the present invention, thereby inducing formation of cytotoxic T cells in a host having a CNS cancer.

In another embodiment, the present invention provides a method of reducing an incidence of a CNS cancer, comprising administering a composition of the present invention. In another embodiment, the present invention provides a method of ameliorating a CNS cancer, comprising administering a composition of the present invention. Each possibility represents a separate embodiment of the present invention.

In one embodiment, the composition is administered to the cells of the subject ex vivo; in another embodiment, the composition is administered to the cells of a donor ex vivo; in another embodiment, the composition is administered to the cells of a donor in vivo, then is transferred to the subject. Each possibility represents a separate embodiment of the present invention.

In one embodiment, the cancer treated by a method of the present invention is breast cancer. In another embodiment, the cancer is an Her2 containing cancer. In another embodiment, the cancer is a melanoma. In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non small-cell lung carcinoma. In another embodiment, the cancer is a glioma. In another embodiment, the cancer is an endometrial carcinoma. In another embodiment, the cancer is a bladder cancer. In another embodiment, the cancer is a head and neck cancer. In another embodiment, the cancer is a prostate carcinoma. In another embodiment, the cancer is Glioblastoma multiforme (GBM). Each possibility represents a separate embodiment of the present invention.

In another embodiment of the methods of the present invention, the subject mounts an immune response against the antigen-expressing tumor or target antigen, thereby mediating the anti-tumor effects.

In one embodiment, a treatment protocol of the present invention is therapeutic. In another embodiment, the protocol is prophylactic. In another embodiment, the vaccines of the present invention are used to protect people at risk for a CNS cancer or metastasis of a cancer such as, but not limited to a breast cancer because of familial genetics or other circumstances that predispose them to these types of ailments as will be understood by a skilled artisan. In another embodiment, the vaccines are used as a cancer immunotherapy after debulking of tumor growth by surgery, conventional chemotherapy or radiation treatment. In another embodiment, the vaccines of the present invention are used in conjunction with alternative cancer therapy programs including debulking growth by surgery, conventional chemotherapy or radiation treatment. Following such treatments, the vaccines of the present invention are administered so that the CTL response to the tumor antigen of the vaccine destroys remaining metastases and prolongs remission from the cancer. In another embodiment, vaccines of the present invention are used to effect the growth of previously established tumors and to kill existing tumor cells. Each possibility represents a separate embodiment of the present invention.

In another embodiment, the vaccines and immunogenic compositions utilized in any of the methods described above have any of the characteristics of vaccines and immunogenic compositions of the present invention. Each characteristic represents a separate embodiment of the present invention.

Various embodiments of dosage ranges are contemplated by this invention. In one embodiment, in the case of vaccine vectors, the dosage is in the range of 0.4 LD₅₀/dose. In another embodiment, the dosage is from about 0.4-4.9 LD₅₀/dose. In another embodiment the dosage is from about 0.5-0.59 LD₅₀/dose. In another embodiment the dosage is from about 0.6-0.69 LD₅₀/dose. In another embodiment the dosage is from about 0.7-0.79 LD₅₀/dose. In another embodiment the dosage is about 0.8 LD₅₀/dose. In another embodiment, the dosage is 0.4 LD₅₀/dose to 0.8 of the LD₅₀/dose.

In another embodiment, the dosage is 10⁷ bacteria/dose. In another embodiment, the dosage is 1.5×10⁷ bacteria/dose. In another embodiment, the dosage is 2×10⁷ bacteria/dose. In another embodiment, the dosage is 3×10⁷ bacteria/dose. In another embodiment, the dosage is 4×10⁷ bacteria/dose. In another embodiment, the dosage is 6×10⁷ bacteria/dose. In another embodiment, the dosage is 8×10⁷ bacteria/dose. In another embodiment, the dosage is 1×10⁸ bacteria/dose. In another embodiment, the dosage is 1.5×10⁸ bacteria/dose. In another embodiment, the dosage is 2×10⁸ bacteria/dose. In another embodiment, the dosage is 3×10⁸ bacteria/dose. In another embodiment, the dosage is 4×10⁸ bacteria/dose. In another embodiment, the dosage is 6×10⁸ bacteria/dose. In another embodiment, the dosage is 8×10⁸ bacteria/dose. In another embodiment, the dosage is 1×10⁹ bacteria/dose. In another embodiment, the dosage is 1.5×10⁹ bacteria/dose. In another embodiment, the dosage is 2×10⁹ bacteria/dose. In another embodiment, the dosage is 3×10⁹ bacteria/dose. In another embodiment, the dosage is 5×10⁹ bacteria/dose. In another embodiment, the dosage is 6×10⁹ bacteria/dose. In another embodiment, the dosage is 8×10⁹ bacteria/dose. In another embodiment, the dosage is 1×10¹⁰ bacteria/dose. In another embodiment, the dosage is 1.5×10¹⁰ bacteria/dose. In another embodiment, the dosage is 2×10¹⁰ bacteria/dose. In another embodiment, the dosage is 3×10¹⁰ bacteria/dose. In another embodiment, the dosage is 5×10¹⁰ bacteria/dose. In another embodiment, the dosage is 6×10¹⁰ bacteria/dose. In another embodiment, the dosage is 8×10¹⁰ bacteria/dose. In another embodiment, the dosage is 8×10⁹ bacteria/dose. In another embodiment, the dosage is 1×10¹¹ bacteria/dose. In another embodiment, the dosage is 1.5×10¹¹ bacteria/dose. In another embodiment, the dosage is 2×10¹¹ bacteria/dose. In another embodiment, the dosage is 3×10¹¹ bacteria/dose. In another embodiment, the dosage is 5×10¹¹ bacteria/dose. In another embodiment, the dosage is 6×10¹¹ bacteria/dose. In another embodiment, the dosage is 8×10¹¹ bacteria/dose. Each possibility represents a separate embodiment of the present invention.

In one embodiment, a vaccine or immunogenic composition of the present invention is administered alone to a subject. In another embodiment, the vaccine or immunogenic composition is administered together with another cancer therapy. Each possibility represents a separate embodiment of the present invention.

In one embodiment, the construct for generating a Listeria strain contains a polylinker to facilitate further subcloning. Several techniques for producing recombinant Listeria are known.

In one embodiment, the construct or nucleic acid molecule is integrated into the Listeria chromosome using homologous recombination. Techniques for homologous recombination are well known in the art, and are described, for example, in Baloglu S, Boyle S M, et al (Immune responses of mice to vaccinia virus recombinants expressing either Listeria monocytogenes partial listeriolysin or Brucella abortus ribosomal L7/L12 protein. Vet Microbiol 2005, 109 (1-2): 11-7); and Jiang L L, Song H H, et al., (Characterization of a mutant Listeria monocytogenes strain expressing green fluorescent protein. Acta Biochim Biophys Sin (Shanghai) 2005, 37 (1): 19-24). In another embodiment, homologous recombination is performed as described in U.S. Pat. No. 6,855,320. In this case, a recombinant Lm strain that expresses E7 was made by chromosomal integration of the E7 gene under the control of the hly promoter and with the inclusion of the hly signal sequence to ensure secretion of the gene product, yielding the recombinant referred to as Lm-AZ/E7. In another embodiment, a temperature sensitive plasmid is used to select the recombinants. Each technique represents a separate embodiment of the present invention.

In another embodiment, the construct or nucleic acid molecule is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in another embodiment, that a stable genomic insertion mutant can be formed but the disadvantage that the position in the genome where the foreign gene has been inserted is unknown.

In another embodiment, the construct or nucleic acid molecule is integrated into the Listerial chromosome using phage integration sites (Lauer P, Chow M Y et al, Construction, characterization, and use of two Listeria monocytogenes site-specific phage integration vectors. J Bacteriol 2002; 184 (14): 4177-86). In certain embodiments of this method, an integrase gene and attachment site of a bacteriophage (e.g. U143 or PSA listeriophage) is used to insert the heterologous gene into the corresponding attachment site, which may be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. Each possibility represents a separate embodiment of the present invention.

In another embodiment, one of various promoters is used to express the antigen or fusion protein containing same. In one embodiment, an Lm promoter is used, e.g. promoters for the genes hly, actA, pica, plcB and mpl, which encode the Listerial proteins hemolysin, actA, phosphotidylinositol-specific phospholipase, phospholipase C, and metalloprotease, respectively. Each possibility represents a separate embodiment of the present invention.

In another embodiment, methods and compositions of the present invention utilize a homologue of a Her-2 chimeric protein or LLO sequence of the present invention. In another embodiment, the methods and compositions of the present invention utilize a Her-2 chimeric protein from a non-human mammal. The terms “homology,” “homologous,” etc, when in reference to any protein or peptide, refer in one embodiment, to a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Methods and computer programs for the alignment are well known in the art.

In another embodiment, the term “homology,” when in reference to any nucleic acid sequence similarly indicates a percentage of nucleotides in a candidate sequence that are identical with the nucleotides of a corresponding native nucleic acid sequence.

Homology is, in one embodiment, determined by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In another embodiment, “homology” refers to identity to a sequence selected from a SEQ ID No: provided herein of greater than 70%. In another embodiment, “homology” refers to identity to a sequence selected from SEQ ID No provided herein of greater than 72%. In another embodiment, the identity is greater than 75%. In another embodiment, the identity is greater than 78%. In another embodiment, the identity is greater than 80%. In another embodiment, the identity is greater than 82%. In another embodiment, the identity is greater than 83%. In another embodiment, the identity is greater than 85%. In another embodiment, the identity is greater than 87%. In another embodiment, the identity is greater than 88%. In another embodiment, the identity is greater than 90%. In another embodiment, the identity is greater than 92%. In another embodiment, the identity is greater than 93%. In another embodiment, the identity is greater than 95%. In another embodiment, the identity is greater than 96%. In another embodiment, the identity is greater than 97%. In another embodiment, the identity is greater than 98%. In another embodiment, the identity is greater than 99%. In another embodiment, the identity is 100%. Each possibility represents a separate embodiment of the present invention.

In another embodiment, homology is determined via determination of candidate sequence hybridization, methods of which are well described in the art (See, for example, “Nucleic Acid Hybridization” Hames, B. D., and Higgins S. J., Eds. (1985); Sambrook et al., 2001, Molecular Cloning, A Laboratory Manual, Cold Spring Harbor Press, N.Y.; and to Ausubel et al., 1989, Current Protocols in Molecular Biology, Green Publishing Associates and Wiley Interscience, N.Y). For example methods of hybridization may be carried out under moderate to stringent conditions, to the complement of a DNA encoding a native caspase peptide. Hybridization conditions being, for example, overnight incubation at 42° C. in a solution comprising: 10-20% formamide, 5×SSC (140 mM NaCl, 14 mM trisodium citrate), 50 mM sodium phosphate (pH 7.6), 5×Denhardt's solution, 10% dextran sulfate, and 20 μg/ml denatured, sheared salmon sperm DNA.

In one embodiment of the present invention, the term “nucleic acids” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment of the present invention.

Protein and/or peptide homology for any amino acid sequence listed herein is determined, in one embodiment, by methods well described in the art, including immunoblot analysis, or via computer algorithm analysis of amino acid sequences, utilizing any of a number of software packages available, via established methods. Some of these packages may include the FASTA, BLAST, MPsrch or Scanps packages, and may employ the use of the Smith and Waterman algorithms, and/or global/local or BLOCKS alignments for analysis, for example. Each method of determining homology represents a separate embodiment of the present invention.

In another embodiment, the present invention provides a kit comprising a reagent utilized in performing a method of the present invention. In another embodiment, the present invention provides a kit comprising a composition, tool, or instrument of the present invention.

The terms “contacting” or “administering,” in one embodiment, refer to directly contacting the cancer cell or tumor with a composition of the present invention. In another embodiment, the terms refer to indirectly contacting the cancer cell or tumor with a composition of the present invention. In another embodiment, methods of the present invention include methods in which the subject is contacted with a composition of the present invention after which the composition is brought in contact with the cancer cell or tumor by diffusion or any other active transport or passive transport process known in the art by which compounds circulate within the body. Each possibility represents a separate embodiment of the present invention.

In another embodiment, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals or organisms. This can be readily carried out by using hybridization probes to identify the same genetic locus in a variety of individuals or organisms. Any and all such nucleotide variations and resulting amino acid polymorphisms or variations that are the result of natural allelic variation and that do not alter the functional activity are intended to be within the scope of the invention.

### Pharmaceutical Compositions

The pharmaceutical compositions containing vaccines and compositions of the present invention are, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of the methods and compositions provided herein, the vaccines or compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound and the inert carrier or diluent, a hard gelating capsule.

In another embodiment, the vaccines or compositions are administered by intravenous, intra-arterial, or intra-muscular injection of a liquid preparation. Suitable liquid formulations include solutions, suspensions, dispersions, emulsions, oils and the like. In one embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

The term “therapeutically effective dose” or “therapeutic effective amount” means a dose that produces the desired effect for which it is administered. The exact dose will be ascertainable by one skilled in the art using known techniques.

The term “about” as used herein means in quantitative terms plus or minus 5%, or in another embodiment plus or minus 10%, or in another embodiment plus or minus 14%, or in another embodiment plus or minus 20%.

The term “subject” refers in one embodiment to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

The following examples are presented in order to more fully illustrate the preferred embodiments of the invention. They should in no way be construed, however, as limiting the to broad scope of the invention.

### EXAMPLES

### Materials and Methods

Oligonucleotides were synthesized by Invitrogen (Carlsbad, Calif.) and DNA sequencing was done by Genewiz Inc, South Plainfield, N.J. Flow cytometry reagents were purchased from Becton Dickinson Biosciences (BD, San Diego, Calif.). Cell culture media, supplements and all other reagents, unless indicated, were from Sigma (St. Louise, Mo.). Her2/neu HLA-A2 peptides were synthesized by EZbiolabs (Westfield, Ind.). Complete RPMI 1640 (C-RPMI) medium contained 2 mM glutamine, 0.1 mM non-essential amino acids, and 1 mM sodium pyruvate, 10% fetal bovine serum, penicillin/streptomycin, Hepes (25 mM). The polyclonal anti-LLO antibody was described previously and anti-Her2/neu antibody was purchased from Sigma.

### Mice and Cell Lines

All animal experiments were performed according to approved protocols by IACUC at the University of Pennsylvania or Rutgers University. FVB/N mice were purchased from Jackson laboratories (Bar Harbor, Me.). The FVB/N Her2/neu transgenic mice, which overexpress the rat Her2/neu onco-protein were housed and bred at the animal core facility at the University of Pennsylvania. The NT-2 tumor cell line expresses high levels of rat Her2/neu protein, was derived from a spontaneous mammary tumor in these mice and grown as described previously. DHFR-G8 (3T3/neu) cells were obtained from ATCC and were grown according to the ATCC recommendations. The EMT6-Luc cell line was a generous gift from Dr. John Ohlfest (University of Minnesota, Minn.) and was grown in complete C-RPMI medium. Bioluminescent work was conducted under guidance by the Small Animal Imaging Facility (SAIF) at the University of Pennsylvania (Philadelphia, Pa.).

### Listeria Constructs and Antigen Expression

Her2/neu-pGEM7Z was kindly provided by Dr. Mark Greene at the University of Pennsylvania and contained the full-length human Her2/neu (hHer2) gene cloned into the pGEM7Z plasmid (Promega, Madison Wis.). The Her2/neu chimeric fragment (ChHer2) and Lm-LLO-ChHer2 construct were generated as described. ChHer2 gene was excised from pAdv138 using XhoI and SpeI restriction enzymes, and cloned in frame with a truncated, non-hemolytic fragment of LLO in the Lmdd shuttle vector, pAdv134. The sequences of the insert, LLO and hly promoter were confirmed by DNA sequencing analysis. This plasmid was electroporated into electro-competent actA, dal, dat mutant Listeria monocytogenes strain, LmddA and positive clones were selected on Brain Heart infusion (BHI) agar plates containing streptomycin (250 μg/ml). In some experiments similar Listeria strains expressing hHer2/neu (Lm-hHer2) fragments were used for comparative purposes. These have been previously described. In all studies, an irrelevant Listeria constructs (Lm-control) was included to account for the antigen independent effects of Listeria on the immune system. Lm-controls were based on the same Listeria platform as ADXS31-164, but expressed a different antigen such as HPV16-E7 or NY-ESO-1. Expression and secretion of fusion proteins from Listeria were tested as described previously. Each construct was passaged twice in vivo as described previously.

### Cytotoxicity Assay

Groups of 3-5 FVB/N mice were immunized three times with one week intervals with 1×10⁸ colony forming units (CFU) of Lm-LLO-ChHer2, ADXS31-164, Lm-hHer2 ICI or Lm-control (expressing an irrelevant antigen) or were left naïve. NT-2 cells were grown in vitro, detached by trypsin and treated with mitomycin C (250 μg/ml in serum free C-RPMI medium) at 37° C. for 45 minutes. After 5 washes, they were co-incubated with splenocytes harvested from immunized or naïve animals at a ratio of 1:5 (Stimulator: Responder) for 5 days at 37° C. and 5% CO₂. A standard cytotoxicity assay was performed using europium labeled 3T3/neu (DHFR-G8) cells as targets according to the method previously described. Released europium from killed target cells was measured after 4 hour incubation using a spectrophotometer (Perkin Elmer, Victor²) at 590 nm Percent specific lysis was defined as (lysis in experimental group-spontaneous lysis)/(Maximum lysis-spontaneous lysis).

Interferon-γ Secretion by Splenocytes from Immunized Mice

Groups of 3-5 FVB/N or HLA-A2 transgenic mice were immunized three times with one week intervals with 1×10⁸ CFU of ADXS31-164, a negative Listeria control (expressing an irrelevant antigen) or were left naïve. Splenocytes from FVB/N mice were isolated one week after the last immunization and co-cultured in 24 well plates at 5×10⁶ cells/well in the presence of mitomycin C treated NT-2 cells in C—RPMI medium. Splenocytes from the HLA-A2 transgenic mice were incubated in the presence of 1 μM of HLA-A2 specific peptides or 1 μg/ml of a recombinant His-tagged ChHer2 protein, produced in E. coli and purified by a nickel based affinity chromatography system (Qiagen, Valencia, Calif.). Samples from supernatants were obtained 24 or 72 hours later and tested for the presence of interferon-γ (IFN-γ) using mouse IFN-γ Enzyme-linked immunosorbent assay (ELISA) kit (BD Biosciences) according to manufacturer's recommendations.

### Tumor Studies in Her2 Transgenic Animals

Six weeks old FVB/N rat Her2/neu transgenic mice (9-14/group) were immunized 6 is times with 5×10⁸ CFU of Lm-LLO-ChHer2, ADXS31-164 or Lm-control. They were observed twice a week for the emergence of spontaneous mammary tumors, which were measured using an electronic caliper, for up to 52 weeks. Escaped tumors were excised when they reached a size 1 cm² in average diameter and preserved in RNAlater (Qiagen) at −20° C. In order to determine the effect of mutations in the Her2/neu protein on the escape of these tumors, genomic DNA was extracted using a genomic DNA isolation kit (Qiagen), and sequenced as described previously.

### Effect of ADXS31-164 on Regulatory T Cells in Spleens and Tumors

Mice were implanted subcutaneously (s.c.) with 1×10⁶ NT-2 cells. On days 7, 14 and 21, they were immunized with 1×10⁸ CFUs of ADXS31-164, LmddA-control or left naïve. Tumors and spleens were extracted on day 28 and tested for the presence of CD3⁺/CD4⁺/FoxP3⁺ Tregs by FACS analysis. Briefly, splenocytes were isolated by homogenizing the spleens between two glass slides in C-RPMI medium. Tumors were minced using a sterile razor blade and digested with a buffer containing DNase (12 U/ml), and collagenase (2 mg/ml) in PBS. After 60 min incubation at RT with agitation, cells were separated by vigorous pipetting. Red blood cells were lysed by RBC lysis buffer followed by several washes with complete RPMI-1640 medium containing 10% FBS. After filtration through a nylon mesh, tumor cells and splenocytes were resuspended in FACS buffer (2% FBS/PBS) and stained with anti-CD3-PerCP-Cy5.5, CD4-FITC, CD25-APC antibodies followed by permeabilization and staining with anti-Foxp3-PE. Flow cytometry analysis was performed using 4-color FACS calibur (BD) and data were analyzed using cell quest software (BD).

### Effect of ADXS31-164 on the Growth of a Breast Cancer Cell Line in the Brain.

Balb/c mice were immunized once a week with 5×10⁸ CFU of ADXS31-164 or an irrelevant Listeria vaccine. Each mouse received three immunizations before tumor implantation. EMT6-Luc cells were grown in vitro then injected into the brain of anesthetized mice at 5,000 cells per mouse. Expression of Her2/neu by EMT6-Luc cells was detected according to a standard Western blot protocol. EMT6-Luc cells produce the enzyme luciferase to and when they metabolize D-Luciferin in vivo, the by-products are photons that are captured ex vivo using a Xenogen X-100 camera and displayed using a heat map. Imaging was performed on anesthetized mice on the indicated days. Pixel intensity is graphed as number of photons per second/cm² of surface area, which is shown as average radiance.

### Statistical Analysis

The log-rank Chi-Squared test was used for survival data and student's t-test for the CTL and ELISA assays, which were done in triplicates. A p-value of less than 0.05 (marked as *) was considered statistically significant in these analyzes. All statistical analysis was done with either Prism software, V.4.0a (2006) or SPSS software, V.14.0 (2006). For all FVB/N rat Her2/neu transgenic studies we used 8-14 mice per group, for all wild-type FVB/N studies we used at least 8 mice per group unless otherwise stated. All studies were repeated at least once except for the long term tumor study in Her2/neu transgenic mouse model.

### Example 1

### Generation of L. monocytogenes Strains that Secrete LLO Fragments Fused to a Tumor Associated Antigen

### Lm Based Construct Expressing Human WT1

Sequence of WT1 protein: Below is the sequence of WT1 spanning 185-517 amino acid residues cloned in Lm based plasmid. The regions in bold refer to epitopes specific for human HLA-A2 (bold) and HLA-A24 (italics).

(SEQ ID NO: 19)
SQASSGQARMFPNAPYLPSCLESQPAIRNQGYSTVTFDQTPSYGHTPSH
HAAQFPNHSFKHEDPMGQQGSLGEQQYSVPPPVYGCHTPTDSCTGSQAL
LLRTPYSSDNLYQMTSQLECMTWNQMNLGATLKGVAAGSSSSVKWTEGQ
SNHSTGYESDNHTTPILCGAQYRIHTHGVFRGIGDVRRVPGVAPTLVRS
ASETSEKRPFMCAYPGCNKRYFKLSHLQMHSRKHTGEKPYQCDFKDCER
RFSRSDQLKRHQRRHTGVKPFQCKTCQRKFSRSDHLKTHTRTHTGKTSE
KPFSCRWPSCQKKFARSDELVRHHNMHQRNMTKLQLAL

### Construction of Listeria Based WT1 Vaccine (LmddA174):

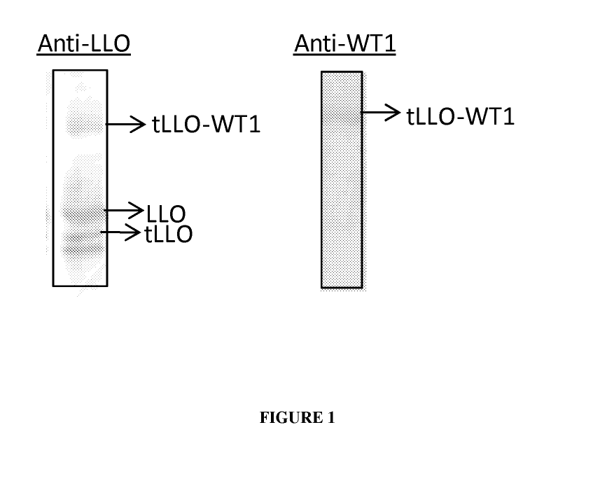

The DNA segment corresponding to the 185-517 amino acid residues of the C-terminus region of human WT-1 gene was cloned Lm based shuttle vector resulting in the plasmid, pAdv174. The plasmid, pAdv174 was transformed in background strain, Lm dal dat actA (LmddA) resulting in LmddA174. The expression of the fusion protein, LLO-WT1 by the strain LmddA174 was analyzed by western blot using anti-PEST and anti-WT1 antibody (FIG. 1). The results show that LmddA174 was expressing and secreting the fusion protein tLLO-WT1 (˜85 kDa).

Lm Based Constructs that Express Her-2/neu Fragments: Construction of ADXS31-164

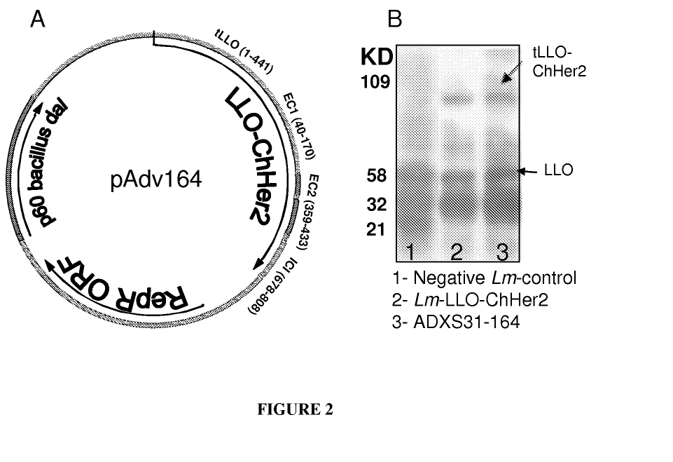

Construction of the chimeric Her2/neu gene (ChHer2) was described previously. Briefly, ChHer2 gene was generated by direct fusion of two extracellular (aa 40-170 and aa 359-433) and one intracellular fragment (aa 678-808) of the Her2/neu protein by SOEing PCR method. The chimeric protein harbors most of the known human MHC class I epitopes of the protein. ChHer2 gene was excised from the plasmid, pAdv138 (which was used to construct Lm-LLO-ChHer2) and cloned into LmddA shuttle plasmid, resulting in the plasmid pAdv164 (FIG. 2A). There are two major differences between these two plasmid backbones. 1) Whereas pAdv138 uses the chloramphenicol resistance marker (cat) for in vitro selection of recombinant bacteria, pAdv164 harbors the D-alanine racemase gene (dal) from bacillus subtilis, which uses a metabolic complementation pathway for in vitro selection and in vivo plasmid retention in LmddA strain which lacks the dal-dat genes. This vaccine platform was designed and developed to address FDA concerns about the antibiotic resistance of the engineered Listeria vaccine strains. 2) Unlike pAdv138, pAdv164 does not harbor a copy of the prfA gene in the plasmid, as this is not necessary for in vivo complementation of the Lmdd strain. The LmddA vaccine strain also lacks the actA gene (responsible for the intracellular movement and cell-to-cell spread of Listeria) so the recombinant vaccine strains derived from this backbone are 100 times less virulent than those derived from the Lmdd, its parent strain. LmddA-based vaccines are also cleared much faster (in less than 48 hours) than the Lmdd-based vaccines from the spleens of the immunized mice. The expression and secretion of the fusion protein tLLO-ChHer2 from this strain was comparable to that of the Lm-LLO-ChHer2 in TCA precipitated cell culture supernatants after 8 hours of in vitro growth (FIG. 2B) as a band of ˜104 KD was detected by an anti-LLO antibody using Western Blot analysis. The Listeria backbone strain expressing only tLLO was used as negative control.

### Lm Based Constructs Expressing Human IL-13Ralpha2 Protein

IL-13Rα2 is a cancer-associated receptor for IL-13 that is structurally different from the physiologic receptor shared with IL-4, IL-13/4-R. IL-13Rα2 can bind only IL-13 and not IL-4 and is abundant on the surface of high-grade glioma cells. IL-13Rα2 is the first plasma membrane receptor ever to be found overexpressed in a vast majority of patients with Glioblastoma multiforme (GBM) and not normal brain cells. Restricted and high level expression of IL-13Rα2 in human gliomas makes this protein an attractive target for vaccine. The known HLA-A2 epitope for IL-13Rα2 which has been described in published studies spans the amino acid residues 345-353. Previously published studies have shown that another analogue of 345-353, HLA-A2 epitope that contains a substitution in amino terminal tryptophan (W) for alanine (A) and carboxy terminal isoleucine (I) for valine (V), known as 1A9V peptide induced enhanced levels of CTL reactivity and protective immunity against an intracranial challenge with IL-13Rα2 expressing syngeneic tumors when compared to the native IL-13Rα2 epitope.

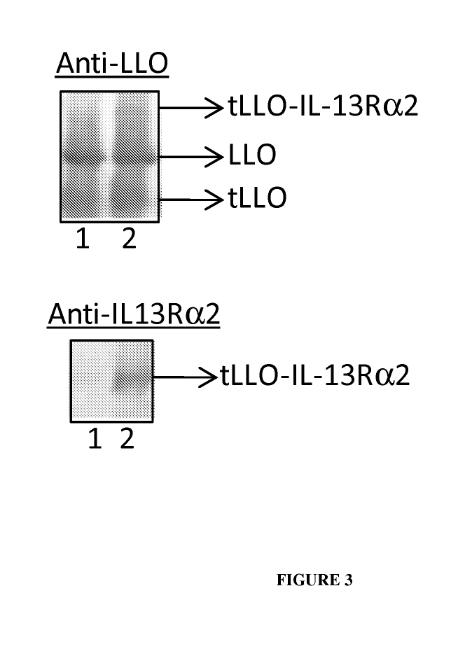

Human IL-13Rα2 is a 380 amino acid single pass type I membrane protein which contains a weakly hydrophobic C-terminus region. Two Lm based constructs were created which express two fragments of human IL-13Rα2 spanning amino acid residues 2-189 (N-terminus) and 180-362 (C-terminus) and thus, were overlapping by 10 amino acids. The C-terminus fragment contains the 1A9V mutations that are present at position 345 and 353 as described above. These two fragments of IL-13Rα2 were successfully cloned in the Lm shuttle plasmid, resulting in plasmids pAdv256 and pAdv257. The plasmids, pAdv256 and pAdv257 were transformed in the LmddA strain resulting in LmddA256 and LmddA257, respectively. Both Lm constructs, LmddA256 and LmddA257 were examined for the expression and secretion of tLLO-IL-13Rα2 fusion protein by western blot with antibodies specific for LLO and IL-13Rα2. The results show that both the constructs LmddA256 and LmddA257 were expressing and secreting the fusion protein tLLO-IL-13Rα2 (˜68 kDa) (FIG. 3), based on the predicted molecular size.

### Sequence of Each Fragment of IL-13Rα2

pAdv256: Contains N-terminus amino acid residues 2-189 of the protein IL-13Rα2

(SEQ ID NO: 20)
AFVCLAIGCLYTFLISTTFGCTSSSDTEIKVNPPQDFEIVDPGYLGYLY
LQWQPPLSLDHFKECTVEYELKYRNIGSETWKTIITKNLHYKDGFDLNK
GIEAKIHTLLPWQCTNGSEVQSSWAETTYWISPQGIPETKVQDMDCVYY
NWQYLLCSWKPGIGVLLDTNYNLFYWYEGLDHALQCVDYI.

pAdv257: Contains C-terminus amino acid residues 180-369 of the protein IL-13Rα2 and mutations at residue 345 (W is replaced by A) and 353 (I is replaced by V). The to region in bold corresponds to the known human HLA-A2 epitope for IL-13Rα2.

(SEQ ID NO: 21)
DHALQCVDYIKADQQNIGCRFPYLEASDYKDFYICVNGSSENKPIRSSY
FTFQLQNIVKPLPPVYLTFTRESSCEIKLKWSIPLGPIPARCFDYEIEI
REDDTTLVTATVENETYTLKTTNETRGLCFVVRSKVNIYCSDDGIWSEW
SDKQCWEGEDLSKKTLLRFALPFGFILVLVIFVTGLL.

### Lm Constructs Expressing Human and Mouse Survivin.

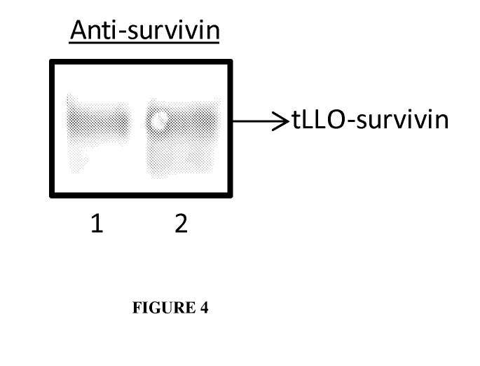

The genes of both human as well as mouse survivin were cloned in Lm based shuttle vector to create constructs plasmids referred as pAdv265 and pAdv266, respectively. The two plasmids, pAdv265 and pAdv266 were transformed in LmddA resulting in LmddA265 and LmddA256. It was observed that both LmddA265 and LmddA266 were expressing and secreting the fusion protein tLLO-survivin (˜64 kDa) as shown by western blot using survivin specific antibody (FIG. 4).

### Sequence of Human Survivin—(2-142 Amino Acids)

(SEQ ID NO: 22)
GAPTLPPAWQPFLKDHRISTFKNWPFLEGCACAPERMAEAGFIHCPTEN
EPDLAQCFFCFKELEGWEPDDDPIEEHKKHSSGCAFLSVKKQFEELTLG
EFLKLDRERAKNKIAKETNNKKKEFEETAKKVRRAIEQLAAMD

### Sequence of Mouse Survivin (2-140 Amino Acids)

(SEQ ID NO: 23)
GAPALPQIWQLYLKNYRIATFKNWPFLEDCACAPERMAEAGFIHCPTEN
EPDLAQCFFCFKELEGWEPDDNPIEEHRKHSPGCAFLTVKKQMEELTVS
EFLKLDRQRAKNKIAKETNNKQKEFEETAKTTRQSIEQLAA

### Listeria Based Constructs Expressing Tumor Antigen CA9

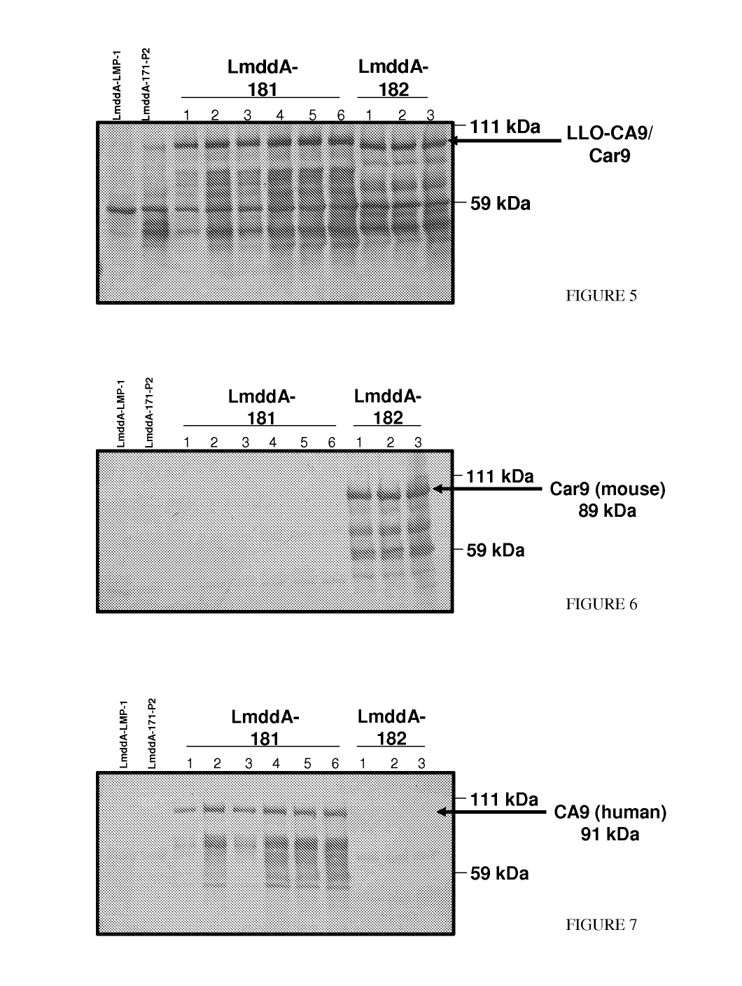

Two Listeria based vaccines expressing human CA9 (human carbonic anhydrase 9) and mouse car9 (mouse carbonic anhydrase 9) were created namely LmddA-181 and LmddA-182, respectively. The vaccine strains were constructed such that the CA9 or Car9 antigen was fused in frame with truncated LLO in the Lm based antibiotic-free shuttle vector. The vaccines were engineered in such a way the N-terminus signal sequence and C-terminus hydrophobic region of both human as well as mouse carbonic anhydrase 9 protein was not included to avoid any expression or secretion problems by Listeria. FIGS. 5-7 show Expression and secretion of tLLO-CA9 and tLLO-car9 fusion proteins by the constructs.

### Protein Sequence of Human CA9 in LmddA181 (38-423 Amino Acid Residues)

(SEQ ID NO: 24)
QRLPRMQEDSPLGGGSSGEDDPLGEEDLPSEEDSPREEDPPGEEDLPGE
EDLPGEEDLPEVKPKSEEEGSLKLEDLPTVEAPGDPQEPQNNAHRDKEG
DDQSHWRYGGDPPWPRVSPACAGRFQSPVDIRPQLAAFCPALRPLELLG
FQLPPLPELRLRNNGHSVQLTLPPGLEMALGPGREYRALQLHLHWGAAG
RPGSEHTVEGHRFPAEIHVVHLSTAFARVDEALGRPGGLAVLAAFLEEG
PEENSAYEQLLSRLEEIAEEGSETQVPGLDISALLPSDFSRYFQYEGSL
TTPPCAQGVIWTVFNQTVMLSAKQLHTLSDTLWGPGDSRLQLNFRATQP
LNGRVIEASFPAGVDSSPRAAEPVQLNSCLAAGDILALVFGLL

### Protein Sequence of Mouse Car 9 in LmddA182 Construct (34-399 Amino Acids)

(SEQ ID NO: 25)
QGLSGMQGEPSLGDSSSGEDELGVDVLPSEEDAPEEADPPDGEDPPEVN
SEDRMEESLGLEDLSTPEAPEHSQGSHGDEKGGGHSHWSYGGTLLWPQV
SPACAGRFQSPVDIRLERTAFCRTLQPLELLGYELQPLPELSLSNNGHT
VQLTLPPGLKMALGPGQEYRALQLHLHWGTSDHPGSEHTVNGHRFPAEI
HVVHLSTAFSELHEALGRPGGLAVLAAFLQESPEENSAYEQLLSHLEEI
SEEGSKIEIPGLDVSALLPSDLSRYYRYEGSLTTPPCSQGVIWTVFNET
VKLSAKQLHTLSVSLWGPRDSRLQLNFRATQPLNGRTIEASFPAAEDSS
PEPVHVNSCFTAGDILALVFGLL.

### Example 2

### Immunogenicity of Constructed Strains

### Immunogenicity of LmddA174 in HLA-A2 Transgenic Mice:

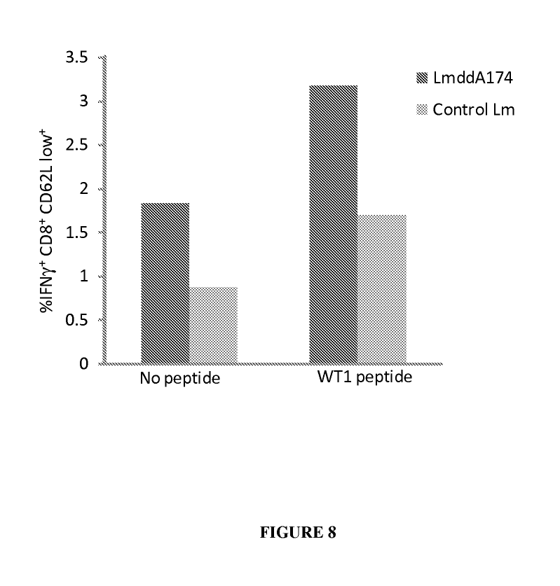

HLA-A2 transgenic mice (3/gp) were immunized with 10⁸ CFU of LmddA174 and Lovaxin C (Listeria control). These mice were boosted on day 14 and 7 days later spleens were harvested. The splenocytes from each group were stimulated in vitro for 7 days using 1 μM of WT1 peptide (RMFPNAPYL) (SEQ ID NO: 26) and 20 U/ml of IL-2 and examined for the induction of IFNγ using intracellular cytokine staining for IFNγ. After 7 days of in vitro stimulation, we observed that 3.17% of CD8⁺ CD62L^{low} cells were secreting IFNγ⁺ in the LmddA174 immunized splenocytes. This was 2 fold higher than the irrelevant Listeria (Lm-LLO-E7) group (FIG. 8). This suggests that LmddA174 construct elicited WT1 specific immune responses in HLA-A2 mice. Since the epitope used in this stimulation is shared by both HLA-A2 and H-2 D^{b}, the elicited responses could not be attributed to a specific MHC haplotype.

### IFN-γ Levels

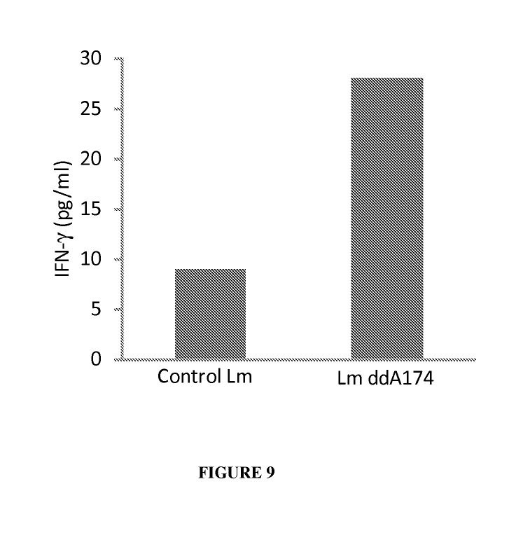

The levels of IFN-γ secreted in the culture supernatants were analyzed using ELISA. It was observed that in case of LmddA174 immunized splenocytes stimulation with WT1 peptide epitope resulted in 28 pg/mL of IFNγ and this was three fold higher than the control Lm group (9 pg/mL) (FIG. 9). This suggests that WT1 specific CD8 T cells were stimulated in vitro in the presence of specific epitope. Since the epitope used in this stimulation is shared by both HLA-A2 and H-2 D^{b}, the elicited responses could not be attributed to a specific MHC haplotype.

### Example 3

### ADXS31-164 is as Immunogenic as Lm-LLO-ChHER2

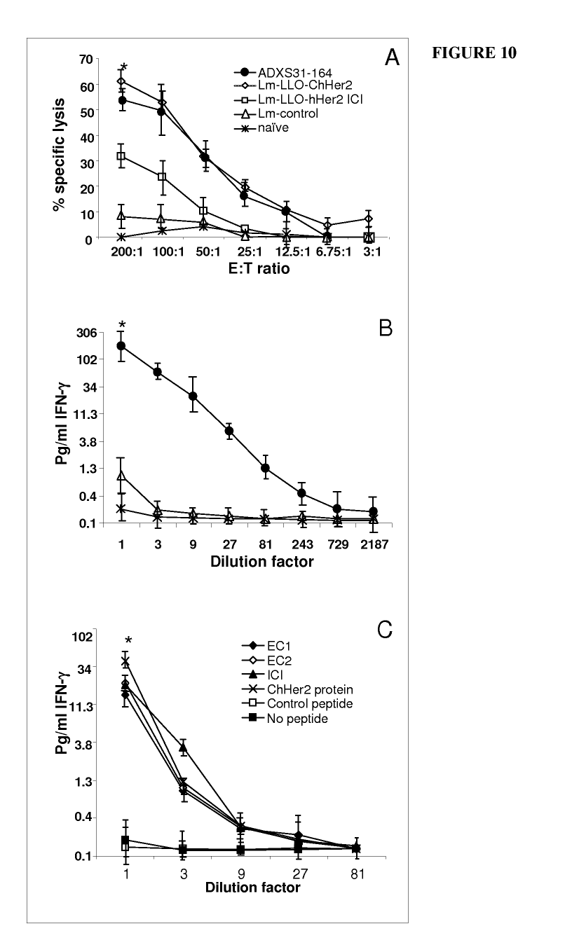

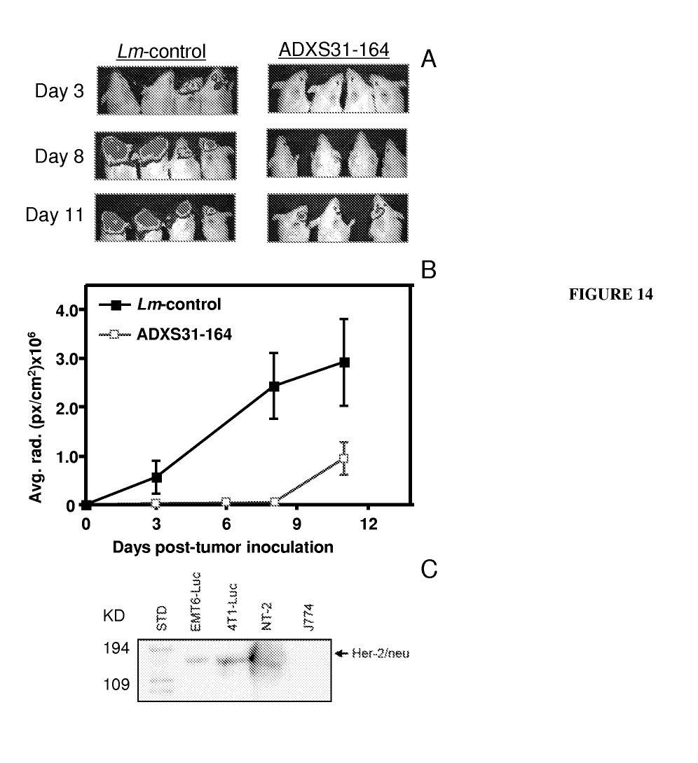

Immunogenic properties of ADXS31-164 in generating anti-Her2/neu specific cytotoxic T cells were compared to those of the Lm-LLO-ChHer2 vaccine in a standard CTL assay. Both vaccines elicited strong but comparable cytotoxic T cell responses toward Her2/neu antigen expressed by 3T3/neu target cells. Accordingly, mice immunized with a Listeria expressing only an intracellular fragment of Her2-fused to LLO showed lower lytic activity than the chimeras which contain more MHC class I epitopes. No CTL activity was detected in naïve animals or mice injected with the irrelevant Listeria vaccine (FIG. 10A). ADXS31-164 was also able to stimulate the secretion of IFN-γ by the splenocytes from wild type FVB/N mice (FIG. 10B). This was detected in the culture supernatants of these cells that were co-cultured with mitomycin C treated NT-2 cells, which express high levels of Her2/neu antigen (FIG. 14C).

Proper processing and presentation of the human MHC class I epitopes after immunizations with ADXS31-164 was tested in HLA-A2 mice. Splenocytes from immunized HLA-A2 transgenics were co-incubated for 72 hours with peptides corresponding to mapped HLA-A2 restricted epitopes located at the extracellular (HLYQGCQVV SEQ ID NO: 27 or KIFGSLAFL SEQ ID NO: 28) or intracellular (RLLQETELV SEQ ID NO: 29) domains of the Her2/neu molecule (FIG. 10C). A recombinant ChHer2 protein was used as positive control and an irrelevant peptide or no peptide as negative controls. The data from this experiment show that ADXS31-164 is able to elicit anti-Her2/neu specific immune response.

### Example 4

### ADXS31-164 was more Efficacious than Lm-LLO-ChHER2 in Preventing the Onset of Spontaneous Mammary Tumors

Anti-tumor effects of ADXS31-164 were compared to those of Lm-LLO-ChHer2 in Her2/neu transgenic animals which develop slow growing, spontaneous mammary tumors at 20-25 weeks of age. All animals immunized with the irrelevant Listeria-control vaccine developed breast tumors within weeks 21-25 and were sacrificed before week 33. In contrast, Listeria-Her2/neu recombinant vaccines caused a significant delay in the formation of the mammary tumors. On week 45, more than 50% ADXS31-164 vaccinated mice (5 out of 9) were still tumor free, as compared to 25% of mice immunized with Lm-LLO-ChHer2. At week 52, 2 out of 8 mice immunized with ADXS31-164 still remained tumor free, whereas all mice from other experimental groups had already succumbed to their disease (FIG. 11). These results indicate that despite being more attenuated, ADXS31-164 is more efficacious than Lm-LLO-ChHer2 in preventing the onset of spontaneous mammary tumors in Her2/neu transgenic animals.

### Example 5

### ADXS31-164 Causes a Significant Decrease in Intra-Tumoral T Regulatory Cells

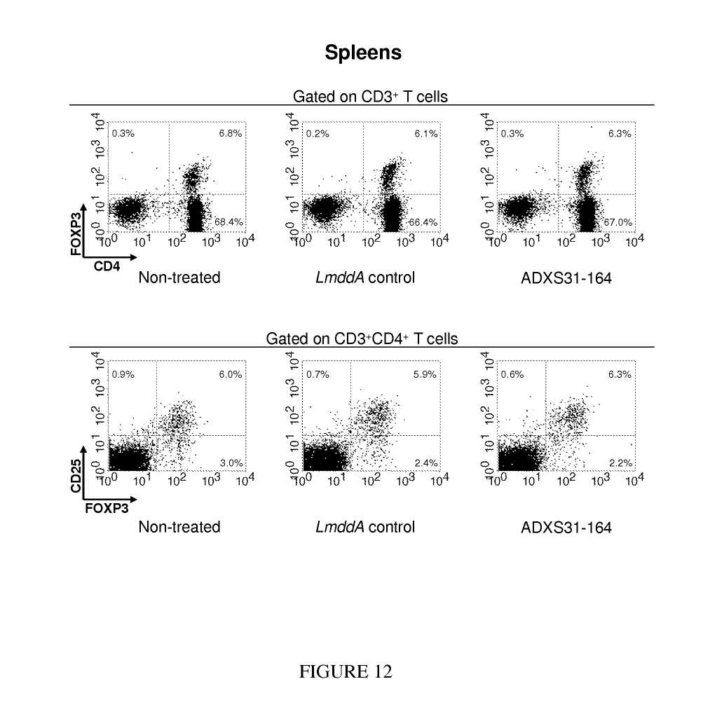

To elucidate the effect of ADXS31-164 on the frequency of regulatory T cells in spleens and tumors, mice were implanted with NT-2 tumor cells. Splenocytes and intra-tumoral lymphocytes were isolated after three immunizations and stained for Tregs, which were defined as CD3⁺/CD4E/CD25⁺/FoxP3⁺ cells, although comparable results were obtained with either FoxP3 or CD25 markers when analyzed separately. The results indicated that immunization with ADXS31-164 had no effect on the frequency of Tregs in the spleens, as compared to an irrelevant Listeria vaccine or the naïve animals (See FIG. 12). In contrast, immunization with the Listeria vaccines caused a considerable impact on the presence of Tregs in the tumors (FIG. 13A). Whereas in average 19.0% of all CD3⁺ T cells in untreated tumors were Tregs, this frequency was reduced to 4.2% for the irrelevant vaccine and 3.4% for ADXS31-164, a 5-fold reduction in the frequency of intra-tumoral Tregs (FIG. 13B). The decrease in the frequency of intra-tumoral Tregs in mice treated with either of the LmddA vaccines could not be attributed to differences in the sizes of the tumors. In a representative experiment, the tumors from mice immunized with ADXS31-164 were significantly smaller [mean diameter (mm)±SD, 6.71±0.43, n=5] than the tumors from untreated mice (8.69±0.98, n=5, p<0.01) or treated with the irrelevant vaccine (8.41±1.47, n=5, p=0.04), whereas comparison of these last two groups showed no statistically significant difference in tumor size (p=0.73). The lower frequency of Tregs in tumors treated with LmddA vaccines resulted in an increased intratumoral CD8/Tregs ratio, suggesting that a more favorable tumor microenvironment can be obtained after immunization with LmddA vaccines. However, only the vaccine expressing the target antigen HER2/neu (ADXS31-164) was able to reduce tumor growth, indicating that the decrease in Tregs has an effect only in the presence on antigen-specific responses in the tumor.

### Example 6

### Peripheral Immunization with ADXS31-164 can Delay the Growth of a Metastatic Breast Cancer Cell Line in the Brain

Mice were immunized IP with ADXS31-164 or irrelevant Lm-control vaccines and then implanted intra-cranially with 5,000 EMT6-Luc tumor cells, expressing luciferase and low levels of Her2/neu (FIG. 14C). Tumors were monitored at different times post-inoculation by ex vivo imaging of anesthetized mice. On day 8 post-tumor inoculation tumors were detected in all control animals, but none of the mice in ADXS31-164 group showed any detectable tumors (FIGS. 14A and B). ADXS31-164 could clearly delay the onset of these tumors, as on day 11 post-tumor inoculation all mice in negative control group had already succumbed to their tumors, but all mice in ADXS31-164 group were still alive and only showed small signs of tumor growth. These results strongly suggest that the immune responses obtained with the peripheral administration of ADXS31-164 could possibly reach the central nervous system and that LmddA-based vaccines might have a potential use for treatment of CNS tumors.

Having described the embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A method of treating the growth of a central nervous system (CNS) cancer in a subject, said method comprising the step of peripherally administering to said subject a composition comprising a live attenuated recombinant Listeria vaccine strain comprising a nucleic acid molecule encoding a polypeptide fused to a tumor antigen, whereby administering said recombinant vaccine strain results in an immune response that effects a therapeutic response across the blood brain barrier of said subject.

2. The method of claim 1, wherein said nucleic acid molecule is in a plasmid in said recombinant Listeria vaccine strain.

3. The method of claim 1, wherein said nucleic acid molecule comprises a first open reading frame encoding a polypeptide, wherein said polypeptide comprises said tumor antigen.

4. The method of claim 3, wherein said nucleic acid molecule further comprises a second and a third open reading frame each encoding a metabolic enzyme, and whereby said metabolic enzyme complements an endogenous gene that is lacking in the chromosome of said recombinant Listeria strain.

5. The method of claim 4, wherein said metabolic enzyme is an amino acid metabolism enzyme.

6. (canceled)

7. (canceled)

8. The method of claim 2, wherein said nucleic acid molecule is integrated into the Listeria genome.

9. (canceled)

10. The method of claim 8, whereby said recombinant Listeria lacks the ActA virulence gene.

11. The method of claim 8, whereby said recombinant Listeria lacks the PrfA virulence gene.

12. The method of claim 1, wherein said live recombinant Listeria vaccine strain is a Listeria monocytogenes-LLO vaccine strain (Lm-LLO vaccine).

13. The method of claim 12, wherein said recombinant strain is the ADXS31-164 strain, LmddA174 strain, LmddA256 strain, LmddA257 strain, LmddA265 strain, LmddA266 strain or the LmddA174 strain.

14. The method of claim 1, wherein said polypeptide is a fusion protein comprising an additional polypeptide selected from the group consisting of: a) non-hemolytic LLO protein or N-terminal fragment, b) a PEST sequence, or c) an ActA fragment, and further wherein said additional polypeptide is fused to said tumor antigen.

15. The method of claim 1, wherein said tumor antigen is PSA, HMW-MAA, HPV-E6, HPV-E7, VEGFR2, Her2/neu, NY-ESO1, survivin, WT-1, IL-2R-α, CA-9.

16. The method of claim 1, wherein said recombinant Listeria strain has been passaged through an animal host.

17. (canceled)

18. (canceled)

19. A method of impeding a growth of a central nervous system (CNS) cancer in a subject, said method comprising the step of peripherally administering to said subject a composition comprising a live attenuated recombinant Listeria vaccine strain comprising a nucleic acid molecule encoding a polypeptide fused to a tumor antigen, whereby administering said recombinant vaccine strain results in an immune response that effects a therapeutic response across the blood brain barrier of said subject.

20. The method of claim 19, wherein said nucleic acid molecule is in a plasmid in said recombinant Listeria vaccine strain.

21. The method of claim 19, wherein said nucleic acid molecule comprises a first open reading frame encoding a polypeptide, wherein said polypeptide comprises said tumor antigen.

22. The method of claim 21, wherein said nucleic acid molecule further comprises a second and a third open reading frame each encoding a metabolic enzyme, and whereby said metabolic enzyme complements an endogenous gene that is lacking in the chromosome of said recombinant Listeria strain.

23. The method of claim 22, wherein said metabolic enzyme is an amino acid metabolism enzyme.

24. (canceled)

25. (canceled)

26. The method of claim 22, wherein said nucleic acid molecule is integrated into the Listeria genome.

27. (canceled)

28. The method of claim 22, whereby said recombinant Listeria lacks the ActA virulence gene.

29. The method of claim 22, whereby said recombinant Listeria lacks the PrfA virulence gene.

30. The method of claim 19, wherein said live recombinant Listeria vaccine strain is a Listeria monocytogenes-LLO vaccine strain (Lm-LLO vaccine).

31. The method of claim 30, wherein said recombinant strain is the ADXS31-164 strain, LmddA174 strain, LmddA256 strain, LmddA257 strain, LmddA265 strain, LmddA266 strain or the LmddA174 strain.

32. The method of claim 19, wherein said polypeptide is a fusion protein comprising an additional polypeptide selected from the group consisting of: a) non-hemolytic LLO protein or N-terminal fragment, b) a PEST sequence, or c) an ActA fragment, and further wherein said additional polypeptide is fused to said tumor antigen.

33. The method of claim 32, wherein said tumor antigen is PSA, HMW-MAA, HPV-E6, HPV-E7, VEGFR2, Her2/neu, NY-ESO1, survivin, WT-1, IL-2R-α, CA-9.

34. The method of claim 19, wherein said recombinant Listeria strain has been passaged through an animal host.

35. (canceled)

36. (canceled)


SEQUENCE LISTING (29 sequences)

SEQ ID NO: 1 - DNA, 1329 bp, Listeria monocytogenes
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgag

SEQ ID NO: 2 - protein, 442 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDL

SEQ ID NO: 3 - protein, 14 aa, Listeria monocytogenes
KTEEQPSEVNTGPR

SEQ ID NO: 4 - protein, 28 aa, Listeria monocytogenes
KASVTDTSEGDLDSSMQSADESTPQPLK

SEQ ID NO: 5 - protein, 20 aa, Listeria monocytogenes
KNEEVNASDFPPPPTDEELR

SEQ ID NO: 6 - protein, 33 aa, Listeria monocytogenes
RGGIPTSEEFSSLNSGDFTDDENSETTEEEIDR

SEQ ID NO: 7 - protein, 17 aa, Streptococcus pyogenes
KQNTASTETTTTNEQPK

SEQ ID NO: 8 - protein, 17 aa, Streptococcus equisimilis
KQNTANTETTTTNEQPK

SEQ ID NO: 9 - protein, 98 aa, Human papillomavirus type 16
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 10 - protein, 105 aa, Human papillomavirus type 16
MHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQ

SEQ ID NO: 11 - protein, 8 aa, Artificial Sequence
  HPV16 E7 antigen
TLGIVCPI

SEQ ID NO: 12 - protein, 9 aa, Artificial Sequence
  HPV16 E7 antigen
LLMGTLGIV

SEQ ID NO: 13 - protein, 10 aa, Artificial Sequence
  HPV16 E7 antigen
YMLDLQPETT

SEQ ID NO: 14 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 15 - protein, 158 aa, Human papillomavirus type 16
MHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQL

SEQ ID NO: 16 - protein, 158 aa, Human papillomavirus type 18
MARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQV

SEQ ID NO: 17 - DNA, 1260 bp, Artificial Sequence
  Her-2 chimeric protein
acccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaa

SEQ ID NO: 18 - protein, 420 aa, Artificial Sequence [sequence omitted]

SEQ ID NO: 19 - protein, 332 aa, Homo sapiens
SQASSGQARMFPNAPYLPSCLESQPAIRNQGYSTVTFDQTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIGDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 20 - protein, 187 aa, Homo sapiens
AFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYI

SEQ ID NO: 21 - protein, 184 aa, Homo sapiens
DHALQCVDYIKADQQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRGLCFVVRSKVNIYCSDDGIWSEWSDKQCWEGEDLSKKTLLRFALPFGFILVLVIFVTGLL

SEQ ID NO: 22 - protein, 141 aa, Homo sapiens
GAPTLPPAWQPFLKDHRISTFKNWPFLEGCACAPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMD

SEQ ID NO: 23 - protein, 139 aa, Mus musculus
GAPALPQIWQLYLKNYRIATFKNWPFLEDCACAPERMAEAGFIHCPTENEPDLAQCFFCFKELEGWEPDDNPIEEHRKHSPGCAFLTVKKQMEELTVSEFLKLDRQRAKNKIAKETNNKQKEFEETAKTTRQSIEQLAA

SEQ ID NO: 24 - protein, 386 aa, Homo sapiens
QRLPRMQEDSPLGGGSSGEDDPLGEEDLPSEEDSPREEDPPGEEDLPGEEDLPGEEDLPEVKPKSEEEGSLKLEDLPTVEAPGDPQEPQNNAHRDKEGDDQSHWRYGGDPPWPRVSPACAGRFQSPVDIRPQLAAFCPALRPLELLGFQLPPLPELRLRNNGHSVQLTLPPGLEMALGPGREYRALQLHLHWGAAGRPGSEHTVEGHRFPAEIHVVHLSTAFARVDEALGRPGGLAVLAAFLEEGPEENSAYEQLLSRLEEIAEEGSETQVPGLDISALLPSDFSRYFQYEGSLTTPPCAQGVIWTVFNQTVMLSAKQLHTLSDTLWGPGDSRLQLNFRATQPLNGRVIEASFPAGVDSSPRAAEPVQLNSCLAAGDILALVFGLL

SEQ ID NO: 25 - protein, 366 aa, Mus musculus
QGLSGMQGEPSLGDSSSGEDELGVDVLPSEEDAPEEADPPDGEDPPEVNSEDRMEESLGLEDLSTPEAPEHSQGSHGDEKGGGHSHWSYGGTLLWPQVSPACAGRFQSPVDIRLERTAFCRTLQPLELLGYELQPLPELSLSNNGHTVQLTLPPGLKMALGPGQEYRALQLHLHWGTSDHPGSEHTVNGHRFPAEIHVVHLSTAFSELHEALGRPGGLAVLAAFLQESPEENSAYEQLLSHLEEISEEGSKIEIPGLDVSALLPSDLSRYYRYEGSLTTPPCSQGVIWTVFNETVKLSAKQLHTLSVSLWGPRDSRLQLNFRATQPLNGRTIEASFPAAEDSSPEPVHVNSCFTAGDILALVFGLL

SEQ ID NO: 26 - protein, 9 aa, Artificial Sequence
  WT1 peptide
RMFPNAPYL

SEQ ID NO: 27 - protein, 9 aa, Artificial Sequence
  mapped HLA-A2 restricted epitopes located at the extracellular domains of the Her2/neu molecule
HLYQGCQVV

SEQ ID NO: 28 - protein, 9 aa, Artificial Sequence
  mapped HLA-A2 restricted epitopes located at the extracellular domains of the Her2/neu molecule
KIFGSLAFL

SEQ ID NO: 29 - protein, 9 aa, Artificial Sequence
  mapped HLA-A2 restricted epitopes located at the intracellular domains of the Her2/neu molecule
RLLQETELV